(19) United States
(12) Patent Application Publication
(10) Pub. No.: US 2026/0115162 A1
(43) Pub. Date: Apr. 30, 2026

(54) COMPOSITION OR ASSOCIATION OF COMPOUNDS PREFERABLY FOR USE IN THE TREATMENT OF NERVOUS DISEASES IN PARTICULAR NEURODEGENERATIVE DISEASES, METHOD FOR THE PREPARATION OF SUCH COMPOSITION OR ASSOCIATION OF COMPOUNDS AND USES THEREOF

(71) Applicant: PHARMACEUTICA SAN MARCO S.R.L., Milano (IT)

(72) Inventor: Matteo BEVILACQUA, Milano (IT)

(21) Appl. No.: 18/841,558

(22) PCT Filed: Mar. 6, 2023
(86) PCT No.: PCT/EP2023/025103

§ 371 Date: Aug. 26, 2024

(30) Foreign Application Priority Data
Mar. 8, 2022 (IT) 102022000004340

(51) Int. Cl. A61K 31/202; A61K 9/00; A61K 9/51; A61K 31/015; A61K 31/19; A61K 31/343; A61K 31/728; A61P 25/22; A61P 25/24; A61P 25/28

(52) U.S. Cl. CPC A61K 31/202; A61K 9/006; A61K 9/51; A61K 31/015; A61K 31/19; A61K 31/343; A61K 31/728; A61P 25/22; A61P 25/24; A61P 25/28

(57) ABSTRACT

Composition or association of compounds comprising: a) the active principle docosahexaenoic acid DHA (C22:6 ω-3 C₂₂H₃₂O₂ MW 328.488) mixed with all or in part, with: b) hyaluronic acid HA₄ tetrasaccharide (C₂₈H₄₄N₂O₂₃ MW 776) in nanoparticles c) β-caryophyllene βCP (C₁₅H₂₄ MW 204.35), d) furanoeudesma-1,3-diene FE (C₁₅H₁₈O MW 214.30), e) β-boswellic acid PBA (C₃₀H₄₈O₃ MW 456.7). preferably for use in the treatment of nervous diseases in particular neurodegenerative diseases, method for the preparation of such composition or association of compounds and uses thereof.

The invention relates to a composition or association of compounds comprising:
a) the active principle docosahexaenoic acid DHA (C22:6 ω-3 C₂₂H₃₂O₂ MW 328.488) mixed with all or in part, with:
b) hyaluronic acid HA₄ tetrasaccharide (C₂₈H₄₄N₂O₂₃ MW 776) in nanoparticles
c) β-caryophyllene βCP (C₁₅H₂₄ MW 204.35),
d) furanoeudesma-1,3-diene FE (C₁₅H₁₈O MW 214.30),
e) β-boswellic acid PBA (C₃₀H₄₈O₃ MW 456.7).
   preferably for use in the treatment of nervous diseases in particular neurodegenerative diseases, method for the preparation of such composition or association of compounds and uses thereof.

The invention is based on the following acquisitions:
a. DHA acts on the lipid system and has its own activities on the Nervous System, and has beneficial effects on learning and memory, on neuroinflammatory processes, on synaptic plasticity and neurogenesis, and on the expression of proteins related to brain cognition; it has demonstrated anti-β-amyloidogenic, antidepressant, hypothalamic-pituitary-adrenal axis activating action.
   DHA is synergistic with β-caryophyllene on the CB2 endocannabinoid system, being a precursor of a diverse repertoire of bioactive lipid mediators, including endocannabinoids.
   DHA is synergistic with ß-boswellic acid in the production of growth factors such as BDNF and in anti-β-amyloidogenic action.
   DHA is synergistic with FE in the opioid μ1-receptor system in the antidepressant action.
   The fact that DHA is a lipid in which the three fat-soluble and poorly water-soluble terpenes βCP, βBA, FE are mixed and solubilised in it is a happy combination and justifies the expectation of an extraordinary synergy.
b. βCP, FE, βBA, DHA are experimentally individually effective on particular anatomical-functional aspects of neurological lesions, but they are almost ineffective on people with advanced neurological diseases due to the difficulty of acting on vast and complex altered substrates.
c. βCP, FE, βBA, DHA being associated and interconnected, interact and integrate among them, producing a surprising synergistic enhancement, prevent and repair functional and tissue damage even in an advanced state while all the therapeutic measures practiced so far give barely significant results only in early stages.
d. βCP, FE, βBA, DHA have complementary mechanisms of action and are interconnected in the endocannabinoids, endorphins, ω3-ω6 polyunsaturated fatty acids and arachidonic acid systems.
e. βCP, FE, βBA, DHA act with synergistic potentiation on inflammation which is the common denominator of many diseases of the Nervous System, particularly neurodegenerative, such as Alzheimer's disease.
f. βCP, FE, βBA, DHA act with synergistic enhancement on interneuronal transmission, cell membrane integrity, neurological damage repair and therefore on mood, memory preservation and cognitive abilities.
g. βCP, FE, βBA, DHA allow a broad and synergistic spectrum of action in neuropsychiatry, such as anxiety, depression, anorexia and bulimia, headache, epilepsy, schizophrenia, dementia and other neurodegenerative diseases.
h. HA₄ contributes to maintaining the anatomo-functional integrity of the Nervous System, allows the recovery of the extracellular matrix and in particular of the perineuronal network, and thus allows the four substances βCP, FE, βBA, e DHA to act on neurons, on glia and synaptic transmission with restorative and regenerating effects.
i. βCP-FE-βBA-DHA-HA₄ association can be a support and complement to therapies with stem cells and active and passive immunology.
j. βCP-FE-βBA-DHA-HA₄ association can be a support, complement and alternative therapy in neurotropic virus diseases, in particular HIV and SARS-COV-2.
k. βCP-FE-βBA-DHA-HA₄ association can be a support, complement and alternative therapy in psychic disorders of female and male sexuality.
l. As nutraceutical substances, the preparation expects the sublingual administration which allows to bypass the gastrointestinal barrier, a much more favorable absorption with a reduced dosage and a more practical administration in neurological people with swallowing problems.
m. The compound derived from the association of the four substances βCP-FE-βBA-HA₄, all together or in part or singly, mixed with DHA, is an object of the invention.
n. The present invention considerably broadens the spectrum of diseases in which it may be effective as a curative, adjuvant, integrative, cognitive enhancer (psychiatric disorders, neurological disorders in particular dementias, demyelinating disorders, movement and cerebellar disorders, brain lobe function and dysfunction, pain, peripheral Nervous System diseases and motor unit dysfunction, stroke, HIV and SARS-COV-2 infections) and as an adjuvant or supplement to immunological and stem cell therapy.

### I DESCRIPTION OF NEUROLOGICAL AND PSYCHIATRIC DISORDERS TREATED BY THE INVENTION

### A Paradigmatic Disease: Alzheimer's Disease (AD)

Alzheimer's disease is the most common form of dementia among people over the age 65, accounting for 50-60% of all dementia cases.

In Italy, dementia affects about 1 million people, in the world about 35.6 million with 7.7 million new cases every year, in the United States 5.5 million and is the sixth cause of death, in 2050 it will affect about sixteen million lives.

Alzheimer's disease is characterized by accumulation of β-amyloid (As) resulting from incorrect processing of the amyloid precursor protein (APP) in plaques outside neurons and by hyperphosphorylation of tau protein which forms neurofibrillary tangles inside neurons. As aggregation, neurofibrillary tangles, other tau protein species such as soluble forms [Kopeikina K J et al. 2012] cause loss of neurons and synapses and gross degeneration in the temporal lobe, parietal lobe, parts of the frontal cortex and cingulate gyrus of the brain.

To date, the molecular mechanisms underlying AD are not fully understood. The chronic inflammatory component has been clearly identified [Heneka M T et al. 2015] with the ability of Aβ aggregates to activate glial cells, thus inducing the release of inflammatory mediators such as ROS, nitric oxide, proinflammatory cytokines, all responsible for neuronal death [Eikelenboom P et al. 1994, Shippy D C and Ulland T K 2020].

Moreover, in AD the monocytes show poor differentiation and only superficial absorption of Aβ, they suffer from apoptosis; macrophage phagocytosis is defective; the levels of cyclooxygenase-2 (COX-2) and intracellular cytokines, including prostaglandins E2 (PGE2), are higher [Fiala M et al. 2005].

The progression of AD is accelerated by an unbalanced deposition/distribution in different regions of the brain of metal ions Zn²⁺, Cu²⁺, Mg²⁺, Mn²⁺, Pb²⁺, Cd²⁺, Hg²⁺, Al³⁺, Fe³⁺. For example, a strong association with FTL (ferritin light chain, protein responsible for the storage of intracellular iron [Shahidehpour R K et al. 2021]) adds oxidative stress, increases aggregation of Aβ and hyperphosphorylation of tau, compromises synaptic functions [Wang L et al. 2020].

More intense dystrophic changes in microglia have recently been highlighted in people with age-related neurodegenerative diseases, including Alzheimer's disease, Down syndrome, Huntington's disease, Lewy body dementia, multiple sclerosis.

Current therapeutic strategies aim at relieving symptoms or slowing progression of the disease. To date, however, no drug used in anti-AD therapy appears to improve prognosis.

Anti-inflammatory drugs, such as NSAIDs, can protect against the onset of AD in genetically predisposed individuals with long-term therapy, but have not given convincing results in AD patients with mild to moderate cognitive impairment [Imbimbo B P et al. 2010] and therefore rationally selected anti-inflammatory drugs should be used [Coray R W and Rogers J 2012].

There is therefore a need to introduce innovative drugs capable of interfering with the pathophysiological mechanisms underlying AD. In addition, that these new preparations may be effective in numerous other morbid neurological conditions marked by inflammation, such as in Parkinson's. In this scenario, growing interest has been focused on the endocannabinoid system (eCB).

### Endocannabinoid System and Neurological Diseases

The eCB system consists of type 1 (CB₁) and type 2 (CB₂), cannabinoid receptors, endogenous lipid ligands such as N-arachidonoylethanolamine (AEA, anandamide) and 2-Arachidonoylglycerol (2-AG), as well as proteins and enzymes involved in their biosynthesis and inactivation. The eCB system is also considered as part of a mechanism that can operate phenotypic and functional morphological changes of the glia and counteract neuroinflammatory processes that occur in neurodegenerative diseases [Grieco M et al. 2021].

There is convincing evidence to support the idea that the eCB system acts as a retrograde signal transmission system, with function of inhibiting the release of neurotransmitters at the presynaptic level. According to the type of cell, this action can last for seconds or hours, significantly affecting function of the neuronal circuit. Endocannabinoids therefore function as neuromodulators and their action takes place in a large number of processes, including pain sensation, stress response, anxiety, appetite and motor learning [Goodman & Gilman, Zanichelli Ed. 2019].

Reduced CB₂ receptor function is associated with severe psychiatric disorders including schizophrenia, major depression, substance abuse [Ishiguro H et al. 2018]. Perturbations in the endocannabinoid system have been found in spinocerebellar ataxia type-3 [Rodriguez-Cueto C et al. 2017], in other autosomal-dominant cerebellar ataxias [Gomez-Ruiz M et al. 2019], in bipolar disorder [Minocci D et al. 2011], in amyotrophic lateral sclerosis [Fernandez-Trapero M et al. 2017], in a mouse model of Niemann Pick disease type C [Oddi S et al. 2019].

Studies conducted both in vitro and in vivo on mouse models have shown that CB₂ receptors mediate inhibition of Aβ-induced neurotoxicity, gliosis and neuroinflammation; that increased expression of CB₂ levels in neuritic plaques found in astrocytes and microglia demonstrates their neuroprotective effect; that pharmacological activation of CB₂ receptors improves memory and cognitive impairments.

### II COMPONENTS OF THE COUPONDS AND THEIR CHARACTERISTICS

### 1. Docosahexaenoic Acid (DHA)

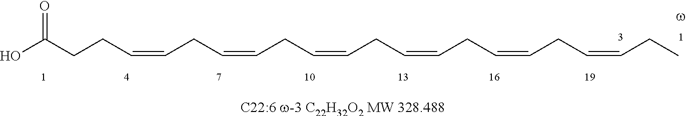 [SMILES: CC/C=C\C/C=C\C/C=C\C/C=C\C/C=C\C/C=C\CCC(=O)O]

Docosahexaenoic acid DHA in the chemical structure is a carboxylic acid with a chain of 22 carbon atoms and 6 double bonds in the cis position; the first double bond is found on the third carbon starting the count from the terminal carbon, hence the term of ω-3.

The brain is the richest organ in lipids (about 50% of the dry weight of the brain). Phospholipids make up more than 60% of total membrane lipids. Brain phospholipids contain two families of ω-3 and ω-6 polyunsaturated fatty acids. The most abundant ω-3 fatty acid is docosahexaenoic acid (accounts for 40% of total membrane phospholipid fatty acids in the brain), followed by eicosapentaenoic acid (EPA, 20:5 ω-3) and docosapentaenoic acid (DPA, 22:5 ω-3), while the main ω-6 fatty acid is arachidonic acid (AA, 20:4 ω-6). DHA and AA are both essential for optimal brain development and function.

Normally, the consumption rate of AA and DHA by the adult human brain has been estimated at 17.8 and 4.6 mg/day, respectively. To maintain normal structure and function, the brain relies on a constant supply of AA and DHA from food through blood [Rapoport S I 2013].

DHA is found in numerous foods of animal and vegetable origin and therefore belongs to category of food supplements. It is present in fish, fish eggs, fish oil, crustaceans, microalgae oil and in foods of plant origin.

Food enrichment with DHA and other long-chain ω-3 has shown beneficial effects on learning and memory [Fairbairn P et al. 2020]; on neuroinflammatory processes [Joffre C et al. 2019]; on synaptic plasticity and neurogenesis [Cao D et al. 2009]; on expression of brain cognition-related proteins, including brain derived neurotrophic factor receptor (BDNFR), N-methyl-D-aspartate receptor (NMDAR) subunits NR2A and NR2B, BDNF protein levels, and presynaptic density-95 (PSD-95) [Hashimoto M et al. 2018] the latter included in our current research.

In Alzheimer's disease, the Aβ₁₋₄₂ peptide has been shown to cause depression in mice. Moreover: in mice, a diet low in ω-3 increases β-amyloid levels [Morgese M G et al. 2020], induces depression and hyperactivation of the hypothalamus-pituitary-adrenal axis and therefore of cortisol (a typical condition of stressful situations, such as anxiety, fear, pain, infections, fasting). Conversely, a diet rich in ω-3 normalizes them [Bove M et al. 2018] and prevents β-amyloid damage [Morgese M G et al. 2018].

In schizophrenia, low erythrocyte levels of EPA and DHA were found, and psychotic symptoms and cognitive deficits improved with corrective dietary intake [Messamore E and McNamara R K, 2016]. Typical of this morbid condition is oxidative stress which induces an increase in phospholipase A2 (PLA2) which causes an overproduction of DHA until its exhaustion [Horrobin D F 1998], modifies physicochemical properties (e.g., fluidity, permeability) of synaptic membranes and causes abnormal neuroinflammation and neurotransmission [Farooqui A A et al. 2007]. Ω-3 supplementation moderates inflammation by significantly reducing the intracellular activity of PLA2 [Smesny S et al. 2014], reconstituting the DHA content of membrane [Hsu M C et al, 2020].

Ω-3 supplementation may also reduce the antipsychotic dose needed to control symptoms, increase antipsychotic tolerability, reduce extrapyramidal side effects, reduce the risk of progression to psychotic disorder, offer a safe and effective strategy for prevention indicated in young people with subthreshold psychotic states [Amminger G P et al. 2010] and improve cognitive performance [Luchtman D W, Song C 2013].

In bipolar disorder, DHA is associated with less neuroticism [Evans S J et al. 2012], moderates inflammation associated with this disease [Chang Y-W et al. 2017], shows efficacy in treatment and is protective against suicidal risk [Evans S J et al. 2011].

DHA has also shown neuroprotective effects in migraine [Soveyd N et al. 2019], depression and anxiety [Larrieu T and Layé S, 2018], in mouse models of spinal cord contusion [Yip P K et al. 2019] and peripheral nerve injury [Gladman S et al. 2012], in spinocerebellar ataxia 38 [Manes M et al. 2017]; raises seizure thresholds and reduces the seizures frequency in epileptic patients [DeGiorgio C M et al. 2015, Reda D M A et al. 2015].

DHA and EPA probably have different metabolic pathways and types of mediators [Dyall S C 2015]. Indeed, only DHA induced changes in memory and significant improvements in verbal fluency [Sinn et al. 2012]; DHA reduced increase in quinolinic acid induced by IL-1β by 78% while EPA was found to be ineffective [Borsini A et al. 2017]; EPA, unlike DHA, worsened clinical conditions and course of amyotrophic lateral sclerosis [Yip P K et al. 2013].

Arachidonic acid, docosahexaenoic acid and eicosapentaenoic acid are precursors of a diverse repertoire of bioactive lipid mediators, including endocannabinoids, suggesting an overlap in neuroprotective effects observed with these different classes of lipids [Larrieu T et al. 2012]. Indeed, mounting evidence suggests an interaction with overlap in protective effects observed with these different lipid classes [Dyall S C. 2017].

DHA toxicology and safety. In mice, DHA is safe up to g 3.2/kg/day.

In children it is safe up to at least 315 mg/day.
In adult humans, harmless up to at least 7.5 g/day [Lien E L 2009]. The US Food and Drug Administration (FDA) defines doses of ω-3 up to 3 g/day “generally considered safe”.
Potential adverse events associated with ω-3 treatment include gastrointestinal disturbances, including nausea, diarrhea, gastroesophageal reflux, belching and, less commonly, vomiting. In most studies, the intake of 600 mg/day was the highest dose employed without side effects [Manes M et al. 2017].

### 2. Hyaluronic Acid Tetrasaccharide (HA₄)

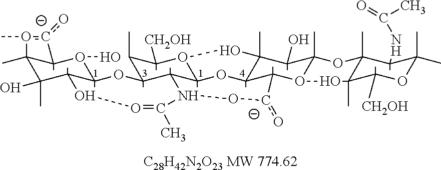

HA tetrasaccharide structure and H bonds in solution. A tetrasaccharide fragment of HA shows five H bonds which help to maintain the double helix. Only in the antiparallel orientation do the participating molecules complement each other so that interactions are optimal. In antiparallel arrays, the acetamido and carboxylate groups are positioned so that H bonds are possible between them. H bonds occur in alternating pairs directed in opposite directions.

The structure is formally equivalent to that of the ß-sheet in proteins, in which pairs of H bonds are arranged in alternating directions between antiparallel polypeptide chains. These cooperative interactions would allow large numbers of HA₄ molecules to specifically aggregate. This structure is relevant for the formation of aggregates between CS chondroitin sulfate and KS keratan sulfate in the extracellular matrix [Scott J E and Heatley F 1999, mod.]. This feature helps to explain the ability of HA to interact with lipids and membranes and suggests how it can interact with itself after HA₄ administration in the Nervous System.

Hyaluronic acid (HA) is a polysaccharide of repeated units of the D-glucuronic acid and N-acetyl-glucosamine disaccharide.

In the most common homeostatic native form:
It is a long polymer chain (up to over 20,000 disaccharides) that can reach a length of 25 nm at full extension, with a high molecular weight between 1,000 and 10,000 kDa. It has a relatively simple structure, but its space-filling properties ensure the preservation of micro-compartments, niches and ion gradients in the brain ultrastructure for optimal cellular function [Melrose J et al. 2021].
It has a significant capacity for hydration and its lack causes a reduction in the volume of the extracellular space (ECS) in the brain. Reduction in ECS volume can initiate or exacerbate epileptic activity in many in vitro models of epilepsy [Perkins K L et al. 2017].
It contributes to maintaining connectivity of neuronal networks in the extracellular matrix [Bikbaev A et al. 2015]. Extracellular matrix (ECM)-neuron interactions can occur mainly in two ways: by hosting growth factors or proteins that bind the growth factor; through cell-extracellular matrix interactions, which can be direct, or receptor-mediated, or by modulating cellular response to growth factors [Rodrigues R S et al. 2019].
It promotes neuronal regeneration, growth, repair and survival [Torigoe K et al. 2011, Wang J et al. 2012] and positively affects neural plasticity, learning and memory [Aydemir C et al. 2006], severely compromised conditions in neurodegenerative disorders, in dementias, in particular in Alzheimer's.
It is a significant component of the perineuronal networks (PNN), as the backbone on which other molecules bind, mainly the lectican chondroitin sulfate proteoglycans.

PNNs are specialized structures that envelop proximal neurons and dendrites, with openings where synaptic inputs contact their underlying cells. Only recently has there been a focus on the role of PNN in physiological functions of the brain, such as learning and memory, as well as in many diseases, including schizophrenia, Alzheimer's disease, stroke, epilepsy, autism, drug addiction and spinal cord injury. Overall, PNNs play key roles in neural development, synaptogenesis, neuroprotection, and experience-dependent synaptic plasticity [Su W et al. 2019].

Hyaluronic acid tetrasaccharide (HA₄) particularly:
It induces (and not other oligosaccharides) differentiation of neuronal cells and oligodendrocyte precursors in the presence of Nerve Growth Factor (NFG) [Termeer C C et al. 2000, Yamanokuchi H 2012]. This means that HA₄ represents an excellent support and complement to future immune or gene therapy in Alzheimer's disease or with stem cells in Parkinson's disease.
It has ability to interact with itself and to aggregate with proteoglycans [Scott J E and Heatley F 1999] and with stabilizing proteins such as tenascins, dynamically regulates and tends to normalize the CNS activity by promoting proliferation of neural stem cells (NSC) in the niches of the subgranular zone of the hippocampal dentate gyrus (SGZ), neural development, synaptogenesis, synaptic plasticity and neuroprotection [Su W et al. 2019]. It is likely that HA₄ acts as a chemo-attractant for different cell types and acts as a trigger to recruit NSCs in synergy with SDF-1α (stromal-derived-factor-1 alpha) bound to it by ionic interactions [Purcell B P et al. 2012].
It increases expression of BDNF which has a positive influence on survival of neurons and stimulates axonal remyelination and damaged spinal cord. Indeed, HA₄ mitigates symptoms of experimental immune encephalitis, inflammatory demyelinating disease, animal model of multiple sclerosis [Winkler C W et al. 2013].
Under stressful conditions, it up-regulates the Hsp72 (Heat shock protein 72) expression and suppresses the cytokines expression, protecting from stress and cell death [Xu H et al. 2002, Kim M et al. 2013].
It interacts with TRPV1 receptors (Transient Receptor Potential Vanilloid subtype-1) non-selective cation channels with high permeability to calcium capable of reducing the nociceptors excitability and therefore both peripheral and central pain [Caires R et al. 2015] and therefore also the central hippocampal neurons excitability, proposing itself as a potential drug against epilepsy [Zhang M et al. 2015].

It should be noted that TRPV1 are also associated with a wide range of functions and behaviors in the central Nervous System, such as fear, anxiety, stress, thermoregulation, pain, and, more recently, synaptic plasticity. This suggests a new role for TRPV1 in areas such as learning and memory, mood, addiction, development [Edwards J G 2014].

It is likely that HA₄ interacts with the k-opioid receptors in concert with the TRPV1 receptors. It is also likely that HA₄ also interacts with TRPA1 receptors (Transient Receptor Potential Ankyrin1) which are co-expressed with TRPV1 and which have complementary functions in pain, neurogenic inflammation, regulating body temperature; are expressed in the same dopaminergic neurons of the substantia nigra, in hippocampal pyramidal neurons, in hypothalamic and locus coeruleus neurons and in various layers of the cortex [Fernandes E S et al. 2011, Aubdool A A et al. 2014, Gentry C et al. 2015].

It is also likely that HA₄ interacts in the same hypothalamic sites with the Toll-like-2, receptors, in particular at the arcuate nucleus level, a brain area that participates in central metabolic regulation by modulating the α-MSH (α-Melanocyte-Stimulating Hormone) and therefore the two pathological forms, obesity and anorexia [Shechter R et al. 2013].

In conclusion, the growing evidence that HA is altered or elevated following CNS insults and during aging implies that HA₄ and HA₄ receptors, are important players in neuroprotection and repair CNS damage, in injury responses, and have a direct roles [Khaing Z Z and Seidlits S K 2015].

It is evident that the hyaluronic acid tetrasaccharide blocks the interactions of the other coarser HA fragments with their receptors in the damaged areas of the Central and Peripheral Nervous System where these fragments have accumulated. This allows the other four substances (βCP, FE, βBA, DHA) to carry out their specific and coordinated action and to act even in advanced stages of neurodegenerative disease.

This is the rationale behind the present invention.

Toxicity. Hyaluronic acid, being a component of the organism, is practically free from toxicity.

In mice: LD₅₀>2,400 mg/kg via os; >4,000 mg/kg subcutaneous; 1,500 mg/kg intraperitoneum.

3. β-cariofillene (βCP)

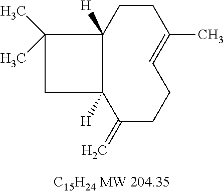

Sesquiterpene β-caryophyllene occurs as a yellow pale liquid with a mixed odor of clove and turpentine. It is abundantly present in essential oils of spices (cinnamon, oregano and black pepper) and in various plants, in particular Cannabis sativa and Copaifera spp.

It is commonly ingested with plant foods and, due to its aromatic characteristics, it is used commercially as a food additive and in cosmetics.

βCP is a lipophilic molecule and is able to cross the blood-brain barrier (BBB) [Elmann A et al. 2009], also because it has a molecular weight below the BBB threshold, which is about 400 Da [Pardridge W M 2012].

It has anti-inflammatory, anti-carcinogenic, antibiotic, antioxidant, anxiolytic, antidepressant, analgesic and local anesthetic effects, and is also not mutagenic, carcinogenic or cytotoxic in cell cultures.

βCP and essential oils containing βCP have neuroprotective potential. Currently, neuroprotective action of βCP has mainly been associated with antioxidant and anti-inflammatory mechanisms [Santos N A G et al. 2017].

βCP showed interesting positive effects in predominantly mouse models in various morbid conditions:
anxiety [Patel S et al. 2017];
depression [Bahi A et al. 2014];
Alzheimer's disease [Cheng Y et al: 2014];
vascular dementia caused by neuro-inflammation [Lou J et al. 2017](but not senile dementia due to mitochondrial dysfunction [Kanojia U et al. 2021]);
Parkinson's disease [Viveros-Paredes J M et al. 2017];
multiple sclerosis [Alberti T B et al. 2017];
Huntington's disease [Sagredo O et al. 2009];
Lennox-Gastaut syndrome and Dravet syndrome [Friedman D et al. 2019];
epilepsy [Tchekalarova et al. 2018];
effects associated with antipsychotics [Navarrete F et al. 2020];
ischemic brain lesions [Chang H J et al. 2013];
functional recovery after spinal cord injury [Latini L et al. 2014];
local anesthetic-like activity [Machado K C et al. 2018]. βCP acts through several mechanisms:
It inhibits pathway leading to the expression of proinflammatory cytokines (IL-1ρ, IL-6, IL-8 e TNF-α, NO, NF-kB, COX-1, COX-2, PGE₂); it suppresses activation of microglia but activates its phagocytic function; it improves oxidative stress and mitochondrial dysfunction [Carlisle S J et al. 2002, Benito C et al. 2008, Wrann C D et al. 2013, Zoppi S et al. 2014, Hashiesh H M et al. 2020, Ullah H et al. 2021].
It is a selective and complete agonist of CB₂ receptors [Gertsch J et al. 2008]. It is not a CB₁ receptor ligand and therefore has no psychoactive effects.
It has analgesic properties involving participation of opioid receptors, mainly μ-opioids, benzodiazepines, serotonin (5-HT_{1A}) [Hernandez-Leon A et al. 2020].
It increases the expression of BDNF [Ferreira F F et al. 2018] a neurotrophin that modulates adult neurogenesis; it is vital for growth, survival, repair, neuronal plasticity, cognitive function, memory and mood [Mu J S et al. 1999].
It increases the expression of PPARs (peroxisome proliferator-activated receptors), specifically PPAR-α, PPAR-ß/δ e PPAR-γ receptors, abundantly expressed in the brain, especially in the striatum, suggesting that they could have a not only anti-inflammatory effect [they inhibit the expression of IL-1β and TNF-α and the activation of NF-kB (Nuclear Factor—kappa-light-chain-enhancer of activated B cells)][Michalik L and Wahli W 2008]), but also anti-degenerative and motor function (Alzheimer's, Parkinson's, Huntington's diseases; multiple sclerosis, stroke, traumatic injuries) [Wójtowicz S et al. 2020, Strosznajder A K et al. 2020].
It induces neuritogenesis and synaptogenesis probably with an independent mechanism of the CB₂ receptor, involving upregulation of proteins related to axonal plasticity (GAP-43, synapsin and synaptophysin) and the activation of the neurotrophic receptor trkA, a member of the family of neurotrophic tyrosine kinase receptors, without binding to the NGF (Nerve Growth Factor) [Santos N A G et al. 2017]. (Axon degeneration is an important finding in many neurodegenerative conditions including stroke, glaucoma, motor neuropathies, amyotrophic lateral sclerosis, Alzheimer's, Parkinson's and Huntington's [Wang J T et al. 2012]).

It has been shown that neurogenesis is permanent in humans with persistence of two neurogenic niches in neurologically healthy subjects up to the ninth decade of life: the subventricular zone and the subgranular zone of the hippocampal dentate gyrus [Boldrini M et al. 2018]. AD patients also have immature progenitor cells although number and maturation progressively decrease as the disease progresses [Moreno-Jimenez E P et al. 2019].

These results demonstrate that memory impairment in AD may be susceptible to new therapeutic strategies. Therefore, there is a need for more integrated, personalized and effective approaches [Moreno-Jimenez E P et al. 2019].

The prospect of using neural stem cells (NSCs) as regenerative therapies is very promising, but several very relevant questions still need to be addressed, in particular how drugs used modulate activities of neural stem cells.

Active and passive immunotherapy has so far only given barely significant benefits in the initial phase of AD.

The focus on cannabinoids as treatment options for various neurological disorders is enormous, particularly when combined with stem cell therapy and immunotherapy [Rodrigues R S et al. 2019]. For example, human mesenchymal stromal cell cultures have been shown to express all components of the endocannabinoid system, suggesting a potential role for the CB₂ cannabinoid receptor as a mediator of their anti-inflammatory properties [Rossi F et al. 2013].

The present invention involving the association of βCP+βBA+FE+DHA+HA₄ has ambition of having overcome the obstacle of ineffectiveness in manifest Alzheimer's disease; in addition, it is proposed as a support and complement to an immunological or stem cell therapy.

Toxicity. The Research Institute for Fragrance Materials (RIFM) has declared β-caryophyllene safe and the molecule has been approved by the Food and Drug Administration and by the European Food Safety Authority as a flavoring agent, food additive, in cosmetics.

It is classified as a category 5 substance (toxic at doses greater than 2,000 mg/kg) in accordance with the guidelines of the OECD (Organization for Economic Co-operation and Development) [Hashiesh H M et al. 2020]. Reports on sub-chronic via os toxicity (700 mg/kg/90d mice) support the safety of β-caryophyllene also for medical products [Schmitt D et al. 2016. Maffei M E 2020].

Toxicity in mice: LD₅₀: 316 mg/kg i.p. (intra peritoneum); with 100 mg/kg i.p. no evidence of pathological changes [Hernandez-Leon A et al. 2020].

4. Furanoeudesma-1,3-diene (FE)

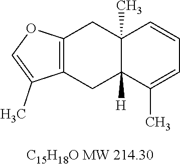 [SMILES: CC1=CC=C[C@]2(C)Cc3occ(C)c3C[C@@H]12]

Myrrh is an aromatic resin with a spicy odor secreted by shrubs of the genus Commiphora of the same Burseraceae family as olibanum. Frankincense and myrrh are intimately intertwined with humanity throughout recorded history, from the incense grains found in the ancient tomb of Pharaoh Tutankhamun to the myrrh-infused brandy blend used to preserve the body of Vice Admiral Horatio Nelson, a 19th-century man, British war hero.

Myrrh is common in tropical northeastern Africa, the Arabian Peninsula and India, and is made up of essential oils, water-soluble gums, and water-soluble resins. In ancient times it was used by the Egyptians for embalming and by the Jews as an ointment. Hippocrates recommended it for plagues, and the Romans used it to treat infections of the mouth and eyes, coughs, and worm infestations. In the Gospel of St. Mark, the “vinum murratum”, wine with myrrh, was offered by the Roman soldiers to Jesus before the crucifixion as amazing, as they used to do in those events.

Myrrh oil has recognized anti-inflammatory, antihistamine, hypolipidemic, hypocholesterolemic, antiatherosclerotic properties; it promotes wound healing with epithelial cell proliferation and is immunostimulating [Malhotra S C et al. 1977, Lata S et al. 1991, Tipton D A et al. 2006, Gebrehiwot M et al. 2015, Al Eid R A 2019, Kuck K et al. 2020]. For example, in mice rendered hyperammonemic, the myrrh resin extract significantly reduced circulating ammonia, liver function markers, TNF-α, glutamine, nitric oxide synthase, soluble guanylate cyclase; suppressed lipid peroxidation; overregulated Nrf2 (nuclear factor erythroid 2-related factor 2) [Mahmoud A M et al. 2017].

There is recent evidence of the analgesic property of myrrh on peripheral pain, such as low back pain [Sureja V et al. 2021], sciatica [Mehta A K and Tripathi C D 2015] and its local anesthetic activity [Dolara P et al. 2000].

The analgesic properties of myrrh have been known since ancient times and depend on the presence of bioactive sesquiterpenes with furanodiene skeletons, and among these furanoeudesma-1,3-diene (FE) is the largest component, by more than 50 percent [Marongiu B et al. 2005, Germano A et al. 2016].

This patent application contemplates the use of furanoeudesma-1,3-diene.

Furanoeudesma-1,3-diene (FE) has an analgesic effect blocked by naloxone, and this indicates an interaction with the μ-opioid receptors of the brain [Dolara P et al. 1996].

The μ-opioid receptors are widely distributed in the central and peripheral nervous systems and in the gastrointestinal tract. At the peripheral level, such as the sciatic nerve, in the mouse model, neuropathic pain can be reduced by activation of peripheral μ-opioid receptors that act on potassium conductance [Stötzner P et al. 2018]. At the supraspinal level, opioid analgesics bind to the μ-receptor located on the GABAergic neurons of the periaqueductal gray matter, the main site of opioidergic analgesia [Hahm E-T et al. 2004, Ghelardini C et al. 2015].

Given that both the orbitofrontal cortex (OFC) and the opioid system regulate reward, motivation and food intake, the role of opioid signaling within the OFC is fundamental for a mechanistic understanding of the sequelae for several psychiatric disorders [Lau B K et al. 2020].

Furanoeudesma-1,3-diene is probably a pure agonist of the opioid receptor subtype μ1 characterized by analgesic action alone, without the μ2 receptors side effects (sedation, respiratory depression, vomiting, dizziness, pruritus, euphoria, anorexia, urinary retention, physical dependence) [Trescot A M et al. 2008, Kong Y et al. 2018]. In fact, the author of the present invention administered by inhalation, by means of an electro-emanator, a terpene preparation also containing sesquiterpenes furanodienes (2.5 mg/2 ml) included in the Rivadol© Turispharma composition, authorized by AIFA, Code AIC 970993491, vaporized with Elettromatt© Turispharma (patent application in Italy filed on 19 Apr. 1996 No. PD96A000097: composition and device inventions by Matteo Bevilacqua) to hundreds of people, obviously with their informed consent and of the attending physician, for various respiratory and neurological diseases without side effects and with excellent documented results [Bevilacqua M, Masson Ed. 2005, pp. 156-167].

The association of FE with βCP e βBA contemplated in the present invention is advantageous in morbid conditions in which there is a reduced availability or reduced efficiency of endogenous μ-opioid receptors, as occurs in numerous neurological diseases with vascular and inflammatory damage. In practice, the FE stimulus can increase the expression of the μ-opioid receptor if the concomitant action of βCP e βBA protects against vascular damage and inflammatory damage and allows the reactivation of the μ-opioid receptor.

For example, it was found that:
the greater expression of μ-opioid receptors is correlated with a greater perfusion of ischemic tissues, as well as with cardioprotection in experimentally induced chronic heart failure and myocardial ischaemia [He S F et al. 2018]. The addition of βBA improves endotolial dysfunction induced by blood stasis and activates the vasodilator intracellular enzyme nitric oxide synthase (eNOS) [Wang M et al. 2015];
a persistent inflammatory nociception reduces the antinociceptive effects of the μ receptor agonist [Jongeling A C et al. 2009] but both βCP and βBA have strong anti-inflammatory properties, as reported in the characteristics of this compound.

Furanoeudesma-1,3-diene has particular indications in the following morbid conditions.

Depression. It is a heterogeneous disorder with patients showing a range of endophenotypes including negative affect, dysphoria, anhedonia, social withdrawal, cognitive impairment, sleep disturbances, changes in appetite and general activity [Akil H et al. 2018].

The μ-opioid receptors are abundantly expressed in the emotional circuit and modulate a variety of functions related to both pleasant and unpleasant emotions, to the gratifying effect of social bond [Nummenmaa L and Tuominen L 2018]; they modulate fear and adverse behavior [Bengoetxea X et al. 2020]; mediate different aspects of opioid-related reward behaviors on distinct neuronal populations [Severino A L et al. 2020].

There is also reduced availability of endogenous μ-opioid receptors in subclinical depression [Nummenmaa L et al. 2020] and a close, bidirectional relationship between opioid receptors and depression in humans [Lutz P E and Kieffer B L 2013].

Anxiety. Even in anxiety there is a reduced availability of endogenous μ-opioid receptors.

The anxiolytic properties of myrrh have also been tested by this inventor in subjects suffering from hyperventilation syndrome [Bevilacqua M, Masson Ed. 2005, p. 127].

Alzheimer's disease (AD). In AD, depression is a risk factor [Green R C et al. 2003] such as to be considered a comorbid condition with negative consequences in patients and healthcare professionals. Depression may precede dementia and tends to occur in up to 50% of AD patients [Modrego P J 2010].

Activation of μ-opioid receptors attenuates neurotoxicity induced by Aß oligomers [Wang Y et al. 2014]. Endomorphine-1 and endomorphine-2, two endogenous opioid peptides with high specificity and affinity for μ-opioid receptors, protect against intracellular toxicity of Aß [Szegedi V et al. 2006, Zhang R S et al. 2015] and improve spatial memory performance: protection is mediated by the induction of estradiol release in hippocampal neurons, which induces upregulation of heat shock protein 70 (Hsp70) [Cui J et al. 2011].

Low doses of morphine, comparable to endogenous brain concentrations, improved long-term memory; high doses did the opposite [Bianchi E et al. 2012].

Multiple sclerosis (MS). In MS, fatigue, depression and pain are highly prevalent and jointly affect more than half of the sufferers [Heitmann H et al. 2020]. A connection between opioids and the immune system is well established [Eisenstein T K 2019]. Endomorphine-1 possesses powerful antinociceptive and anti-inflammatory properties: it increases the secretion of the anti-inflammatory cytokine interleukin (IL)-10 and suppresses the secretion of the pro-inflammatory cytokines IL-12 and IL-23; improves peripheral inflammatory pain and reduces a localized inflammatory response. Endomorphine-2 inhibits release of inflammatory mediators, such as tumor necrosis factor (TNF)-α and IL-12; attenuates chemotaxis and phagocytosis of macrophages [Dworsky-Fried Z et al. 2021].

Parkinson's disease (PD). In PD, depressive disorders are common and may even precede the onset of motor symptoms; they influence many clinical aspects of the disease; they are often associated with other neuropsychiatric symptoms and with late-stage complications such as dementia; they have a negative impact on quality of life, on motor and cognitive deficits, on functional disability [Marsh L 2013, Assogna F et al. 2019]. Since dopamine is necessary for the formation of endogenous morphine in the mammalian brain [Neri C et al. 2008] a deficiency of endorphins in Parkinson's disease can be hypothesized.

Physical exercise also improves painful symptoms with an increase in the expression of μ-opioid receptors in the thalamus [Binda K H et al. 2021]. In addition, μ-opioid receptor agonists have a protective effect against cell damage [Eftekhar-Vaghefi S et al. 2015], alleviate dyskinesia [Bezard E et al. 2020].

Huntington's disease (HD). In 25 people with HD, the levels of forebrain proenkephalin (pENK) were reduced in a manner closely related to severity of the disease [Niemela V et al. 2020]. In 48 HD patients, blood and cerebrospinal concentrations of endorphins were significantly reduced [Nikol'skaia N N et al. 1996].

An upregulation of the μ-opioid-1 receptor in the caudal region of the striatum was found in a mouse model of HD [Morigaki R et al. 2020]. According to another mouse experiment, the striatal overexpression of pENK had beneficial effects on behavioral symptoms: delay in onset of decline in muscle strength; reduction of hooking; improvement of fast motor activity, short-term memory and recognition; normalization of anxious behavior. Consequently, it is likely that upregulation of the striatal encephalin may play a key role in alleviation of disease symptoms in the early phase of HD [Bissonette S et al. 2013].

In addition, reduced levels of the Brain-Derived Neurotrophic Factor (BDNF) neurotrophin anticipate the onset of motor dysfunction and produce more severe uncoordinated movements. Therefore, administration of exogenous BDNF can delay or stop the progression of the disease [Canals J M et al. 2004].

Major Depressive Disorder (MDD). It is one of the most widespread psychiatric disorders. Despite the widespread use of drugs to treat depression, only 35% of patients achieve complete remission of symptoms. Conventional antidepressants require 4-6 weeks of administration before therapeutic efficacy begins, during which time patients continue to experience disabling levels of depression and in some cases relentless suicidal ideation [Browne C A and Lucki 12019].

Currently, nearly all Food and Drug Administration (FDA) approved pharmacotherapies for MDD depression share a common mechanism of action, increased monoaminergic neurotransmission of norepinephrine, dopamine, and serotonin. An emerging pathway is the modulation of endogenous opioid tone, unregulated in depression, for the development of new drugs [Peciña M et al. 2019].

This model is based upon recent findings of opioid modulation of human social learning, bonding and empathy in relation to affiliative and protection tendencies. Fundamental to the model is that the μ-opioid system reinforces socially affiliative or protective behavior in response to positive and negative social experiences with long-term consequences for social behavior and health [Meier I M et al. 2021] and can be an important factor contributing to psychological and psychosomatic resilience to stress, fear, anxiety, anhedonia [Henry M S et al. 2017].

In addition, activation of inflammatory markers has been found in bipolar disorder with abnormal mood states [Fiedorowicz J G et al. 2015], which can also be modulated by the present invention.

Schizophrenia. It is characterized by a pharmacological block of the μ-opioid system which induces conditioned place aversion and reduces social novelty preference.

The stimulation of μ-opioid receptors increases motivation to seek reward, social acceptance, food palatability and hedonic evaluation [Ashok A H et al. 2019].

Schizophrenia and bipolar disorder are conceptualized as dichotomous disorders and as belonging to a continuum in which psychotic depression and schizophrenic disorder fall into two extremes [Stahl S T. Essential Psychopharmacology. Cambridge Ed. 2021: 249].

Suicide. In people who died of suicide—suffering from schizophrenia, major depressive disorder, bipolar disorder—the availability of the μ-receptor for endogenous opioids is decreased [Scarr E et al. 2012].

Autism. It is characterized by a decrease in μ-opioid receptors with severe impairment of social interactions [Pellissier L P et al. 2018].

In mice, opioid neurotransmission of the nucleus accumbens (NAc) in social play behavior was studied: NAc μ-opioid receptor stimulation is an important neural mechanism for attribution of positive value to social interactions in adolescent mice. Altered N Ac μ-opioid receptor function may underlie social impairments in psychiatric disorders such as autism, schizophrenia, or personality disorders [Trezza V et al. 2011].

Anorexia and bulimia. The brain's endogenous opioid system has been implicated in eating behavior.

The expression of the μ-opioid receptor in the insular cortex is decreased in bulimia nervosa and is inversely correlated with fasting behavior [Bencherif B et al. 2005].

Agonists of μ-opioid receptors increase food intake while antagonists inhibit it [Beckman T R et al. 2009].

Commiphora myrrh resin extract in mice, after a diet rich in fat, reduced food intake and body weight, improved hyperglycemia, dyslipidemia, ketonemia, lipid peroxidation of liver tissues; restored architecture of the liver tissue; improved the protein expression of leptin, adiponectin and activity of hepatic glutathione reductase [Orabi S H et al. 2020].

Headaches. Opioid agonists have been used for many years to treat all forms of headache, including migraine. The μ-opioid receptors (and not the δ- and k-receptors) modulate nociceptive neurotransmission [Williamson D J et al. 2001, Storer R J et al. 2003].

Research in the previous decades has produced more than 50 new analgesics. However, these analgesics do not have sufficient efficacy to demonstrably replace the use of opioids or nonsteroidal anti-inflammatory drugs for the treatment of pain. All newly approved and candidate drugs show that although they have completely new mechanisms of action, they have demonstrated the same persistent problems: relatively low therapeutic advantage over previous treatment and narrow spectrum of use in different types of pain, compared with opioids or NSAIDs [Kissin I 2021].

Note that botulinum toxin A approved for the treatment of chronic migraine may be associated with endogenous opioid system activity involving the μ-receptor [Drinovac V et al. 2013]. Commiphora myrrh itself is still proposed today as an alternative treatment in migraine prophylaxis [Tonini M C and Giordano L 2018].

Epilpsy. It is a common neurological disorder, about 1% of the world population suffers from this disease. One of the most common forms is epilepsy of the anterior cingulate cortex (ACC), a variety of frontal lobe epilepsy, refractory epilepsy for which finding an alternative therapeutic approach is very important [Chang W P and Shyu B C 2014].

Both glutamatergic and GABAergic signaling contribute to epileptiform synchronization leading to generation of ictal events in ACC [Avoli M et al. 1996, Jang C G et al. 2001].

It has been shown that μ-opioid receptors are involved in the epileptic synchronization mechanism in ACC seizures.

The selective agonist of the opioid receptor and to a lesser extent the δ receptor agonist suppressed epileptiform activity; the k receptor agonist, no [Chang W P and Shyu B C 2014, Panahi Y et al. 2017].

Therefore, activation of μ-opioid receptors may represent a future prospect in control of cingulate epilepsy [Panuccio G et al. 2009].

Conclusion. FE, a selective μ-opioid agonist, is an essential component of the present invention, due to its analgesic, anti-anxiety, antidepressant, protective effects on prosociality and positive mood, appetite disorders, headaches, epilepsy, and in numerous other morbid conditions such as AD, MS, PD, HD, MDD, schizophrenia, and autism.

It is safe and effective, with no risk of abuse or addiction.

FE likely behaves as a positive allosteric modulator of μ1-receptors that enhances the efficacy of opioids but without their adverse effects [Pryce K D et al. 2021].

Toxicity and side effects. Myrrh is considered a natural and safe substance and has been approved by the Food and Drug

Administration [Ford R A et al. 1992]. The myrrh extract was tested:
in humans, 10 mg/kg/os/6 days, [Sheir Z et al. 2001]; 100 mg/d/os/6 months, well tolerated [De Leo V et al. 2019];
in mice: LD₅₀ 3,000 mg/kg/os. 1,200 mg/kg/os, non-toxic [Gebrehiwot M et al. 2015]; −500 mg/kg/os, well tolerated [Massoud A M A et al. 2004, Lamichhane R et al. 2019].

Myrrh essential oil and its constituents, including FE, via os was tolerated without side effects in 184 volunteers [Germano A et al. 2017].

Topical application caused persistent skin irritation in mice [Saeed M A, Sabir A W 2004] and contact dermatitis in humans [Gallo R et al. 1999].

5. β-boswellic acid ((3BA)

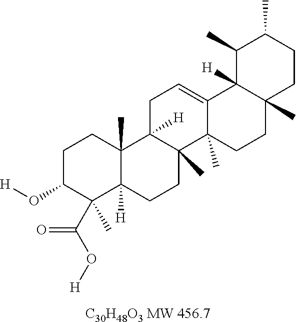

The olibanum, or frankincense, a resin produced by Boswellia plants of the Burseraceae family, has been known since ancient times for its healing properties.

The Boswellia genus is divided into about fifteen species. The plant is native to the Persian Gulf in the Indian Ocean and is cultivated in numerous countries such as southern Arabia, Somalia, Ethiopia, Eritrea, Sudan and Kenya. The Boswellia serrata, most commonly used, is grown in India.

The olibanum, and therefore its components, have been used since ancient times, and still today, as food supplements.

The Boswellia resin use has been proposed for various inflammatory conditions, such as rheumatoid arthritis, osteoarthritis, chronic colitis, ulcerative colitis, Crohn's disease and bronchial asthma [Ammon H P 2016].

According to most Authors, the therapeutic effects of boswellic acids can be attributed to immunomodulatory, anti-inflammatory, antioxidant activity and to elimination of senescent cells.

Furthermore, the genus Boswellia, which includes about 20 species, has been studied as a new candidate for neurodegenerative disorders, including Alzheimer's disease [Rajabian A et al. 2020] and Parkinson's disease [Doaee P et al. 2018].

The phytochemical content of the resins of the various Boswellia species is dependent on the botanical origin and consists of triterpenes (30-60%), (such as α- e β-boswellic acids, lupeolic acid), essential oils (5-10%), polysaccharides.

Boswellic acids (BA) influence the cellular defense system through interaction with production/release of cytokines. Therefore, the BAs inhibit the NF-kB activation which is a product of neutrophilic granulocytes. Consequently, a down-regulation of TNF-α and a decrease in IL-1, IL-2, IL-4, IL-6 e IFN-γ, which are proinflammatory cytokines, by boswellic acids have been reported [Cavaillon J M 2001]. It was found that the suppression of the classical pathway of the complement system is due to inhibition of the conversion of C3 to C3a and C3b.

Boswellic acids inhibit key molecular targets and signaling pathways such as 5-lipoxygenase/cyclooxygenase, Nrf2, NF-kB, cholinergic, beta-amyloid (As) and neurofibrillary tangle formation (NFT) that are involved in the progression of AD. [Gomaa A A et al. 2021, Siddiqui A et al. 2021].

Olibanum in elderly men with moderate mental status has facilitated acquisition and retention of the explicit motor memory [Asadi E et al. 2019]; improved learning ability and cognitive function in mice made epileptic [Jalili C et al. 2014].

This patent application contemplates the use of β-boswellic acid.

β-boswellic acid (βBA), in line with these data, has antioxidant and anti-inflammatory properties [Schmiech M et al. 2019] and has recently been tested as a potential therapeutic drug for AD. Indeed βBA:
In vitro, it inhibits mPGES-1 (prostaglandina E₂ synthase-1); it inhibits catG (cathepsin G), IC₅₀ 0.8 μM, more effective than AKBA (acetyl-11-keto-beta-boswellic acid) [Tausch L et al. 2009]; lipoxygenase (less effectively than AKBA) [Koeberle A et al. 2018, Siemoneit U et al. 2010]; LPS (lipolysaccharide) [Henkel A et al. 2012]; it suppresses the mobilization of Ca²⁺ in human platelets induced by catG [Tausch L et al. 2009]; it inhibits cyclooxygenases, in particular COX-1 [Siemoneit 2008]; it has other mechanisms underlying the anti-inflammatory action in addition to those mentioned above.
It improves memory by acting on the expression of the CREB-1 and CREB-2 genes [Jebelli A et al. 2018].
It interacts with the Tau protein, forming a stable βBA-Tau complex with hydrophobic bonds and stabilizing microtubules [Haghaei H et al. 2019].
It has an anti-neurodegenerative effect by reducing the hyperphosphorylation of tau protein and fibrillar acid protein while increasing expression of the reelin protein with improved learning and memory [Shasaltaneh M D et al. 2021].
It has an effect on polymerization kinetics of microtubules and therefore on axonal growth and branching of hippocampal neurites [Karima O et al. 2010, Karima O et al. 2012].
It can mitigate endothelial cell damage in a blood stasis model and protect endothelial cells from cell death induced by oxygen and glucose deprivation, also increases the intracellular level of NO and cGMP (cyclic guanosine 3′,5′-monophosphate) resulting in vasodilation [Wang M et al. 2015]. This same mechanism can be exploited to protect against microcirculatory damage present in other diseases without amyloid deposition, such as amyotrophic lateral sclerosis/parkinsonism-dementia/Pick's disease [Buée L et al. 1997]. It is interesting to note that βBA significantly prolonged thrombin (TT), prothrombin (PT) and partial thromboplastin (APTT) time and reduced fibrinogen level (FIB) compared with a model group and demonstrated the role of βBA in modulating the parameters of plasma coagulation in a dose-dependent manner [Wang M et al. 2015], synergistic with aspirin.
It has an anticholinesterase effect [Byler K G and Setzer W N 2018] with beneficial effects in humans on Alzheimer's dementia [Tajadini H et al. 2015].

However, AD is a complex neurodegenerative disease characterized, even in its early stage, by mood swings. In particular, it has been shown that a depressive state can largely precede cognitive decline [Geerlings M I et al. 2008, Geerlings M I et al. 2000] and this event may be associated with an increase in levels of soluble neurotoxic species of AP [Ledo J H et al. 2016].

In this regard, we have previously shown that an intracerebroventricular injection of soluble Aβ₁₋₄₂ generates depression-like behavior in mice accompanied by an altered monoamine content in the prefrontal-cortical cortex (PFC) and in areas of the hippocampus (HIPP), increased glial activation and neuroinflammation [Colaianna M et al. 2010, Bove M et al. 2018, Morgese M G et al. 2018]. Indeed, much evidence suggests that some types of depression are associated with increased inflammatory status [Bauer M E, Teixeira A L 2019].

βBA, given via os, has a significantly higher bioavailability than AKBA considered the most active of the boswellic acids in vivo and in vitro experiments (See Table 1).

In fact, in mice, the blood concentration of AKBA is also reduced by 80 times compared to the dose introduced orally and by more than twice when crossing the blood-brain barrier [Gerbeth K et al. 2013]. In practice, 80 mg of AKBA administered to 13 patients did not cross the intestinal mucosal barrier (15.5 ng/ml of blood were found in only one of them) [Gerbeth K et al. 2011].

Instead, βBA was significantly less reduced than AKBA in passing into the blood. This is likely due to the lack of the keto group and the lower molecular weight of βBA (457 Da) compared to AKBA (512 Da). Therefore, the poor digestive absorption of AKBA makes this terpene much more suitable for the treatment of inflammatory diseases of the intestine [Catanzaro D et al. 2015] rather than for neurological disorders.

The pharmacokinetics of βBA was studied in young healthy volunteers both fasted and after hyperlipidic meals [Sterk V et al. 2004] and demonstrates βBA has a more favorable bioavailability than AKBA.

Indeed, βBA has a concentration in the blood:
at least 6 times higher than AKBA both in the fasting condition and with a hyperlipidic meal;
increased by 6 times after hyperlipidic meal so that the plasma/os ratio passes from 0.09 to 0.55.

Therefore, it is foreseeable that the compound object of this invention, which contemplates the association with DHA fatty acid, improves even more the βBA absorption, of the compound the least absorbable terpene (“Lipinski's rule of five” 1 [Vijayarani K R et al. 2020]).

βBA appears not to undergo blood-brain barrier. According to the data of Gerbeth K et al. 2013, brain levels of AKBA were very low, resulting 0.4 per AKBA in the brain/plasma ratio (but 0.81 according to Weber C C et al. 2006). In contrast, the § BA concentration in the brain was even higher than that found in the blood with 32% increase (See Table 1).

| | Authors | | | | | |
|---|---|---|---|---|---|---|
| | Sterk V et al. 2004 | | Gerbeth K et al 2011 | | Gerbeth et al. 2013 | |
| Experimentation | healthy volunteers | | patients | | mice | |
| ng/g o ng/ml | βBA | AKBA | βBA | AKBA | βBA | AKBA |
| via os | 2,048 | 410 | 31,950 | 1,148 | 28,000 | 7,640 |
| in the plasma | 1,120 hyperlipidic | 28 hyperlipidic | 2,236 | 0-15 | 725 | 93 |
| | fed conditions | fed conditions | | | | |
| | 188.2 fasted | 6 fasted | | | | |
| | conditions | conditions | | | | |
| in the brain | | | | | 1,066 | 37.5 |
| Δos /plasma | 1.82 hyperlipidic | 15 hyperlipidic | 14.3 | not | 39 | 82 |
| | fed conditions | fed conditions | | quantifiable | | |
| | 10.9 fasted | 68 fasted | | | | |
| | conditions | conditions | | | | |
| Δos/brain | | | | | 26 | 204 |
| Δplasma/brain | | | | | 0.68 | 2.5 |

Table 1 highlights two results:
the significant difference via os βBA and AKBA absorption, mainly due to the intestinal mucosa barrage according to the molecular weight (βBA = 457 Da, AKBA = 512 Da);
the notable increase in intestinal βBA (6 times more) and AKBA (4.5 times more) induced by a hyperlipidic meal.

Sublingual administration allows for greater bioavailability. The present invention precisely contemplates this method of administration, the effectiveness of which for the first time in literature has been demonstrated by the same author and Coll. [Morgese M G et al. 2021].

According to the present experiment, with sublingual administration the reduction in blood concentration is around 45% (Δos/plasma=1.81), while with via os administration the blood rate was 67% lower than at the assumed dose (Δos/plasma=2.97) with a route sublingual gain of 40%. In short, the sublingual route allows for greater absorption comparable to the enhancement obtained with hyperlipidic meal βBA oral intake.

βBA in comparison to the other boswellic acids has a more marked anti-inflammatory activity [Du Z et al. 2015] and therefore greater therapeutic efficacy in neurological diseases with an inflammatory imprint.

Likely, this also progressively produces a better diffusion from the blood to the CNS through the blood-brain barrier (BBB) and the blood-cerebrospinal fluid epithelial barrier (BCSFB) precisely where the damage in neurodegenerative diseases, such as Alzheimer's, is most severe. In fact, the BBB (which segregates the cerebral interstitial fluid, ISF, from the circulating blood and is located at the level of the cerebral capillaries) and the BCSFB (which is located in the choroid plexus and separates the blood from the cerebrospinal fluid, CSF, that flows in the subarachnoid space) are characterized by early inflammatory engagement in these diseases due to the convergence of different cell types: endothelial cells (BBB), epithelial cells (BCSFB), pericytes, astrocytes and microglia (perivascular macrophages).

Pharmacokinetics of Boswellia serrata extract (BSE). Peak plasma BSE levels were achieved at 4.5±0.55 h. Concentration decreased with a mean elimination half-life of 5.97±0.95 h. The apparent distribution volume was on mean of 142.87±22.78 l and the plasma clearance of 296.10±24.09 ml/min. The AUC₀₋∞ was 27.33×10⁻³±1.99 μmol/ml/h.

Conclusion: The half-life elimination of nearly six hours suggests that the drug should be administered orally possibly at six-hour intervals. The plasma concentration reaches steady state after approximately 30 hours.

BSE is a safe and well tolerated drug for oral administration. No adverse effects were observed with this drug when administered as a single dose in 333 mg (βBA=18.51%=61.63 mg) [Sharma S et al. 2004, Furtado N A J et al. 2017].

Toxicity. Boswellia is often included in multi-ingredient dietary supplements, some of which have been implicated in liver damage, but a specific contribution of Boswellia to damage could not be established. Likelihood Score: E (unlikely cause of clinically evident liver injury). [PubChem—National Library of Medicine].

The frequency of Boswellia hypersensitivity reactions is also unknown.

### III INTERACTIONS BETWEEN DHA, HA₄, βCP, FE, βBA

### A. Interactions Between DHA e βCP

1. There is a complex interconnection and interaction between arachidonic acid AA, docosahexaenoic acid DHA, eicosapentaenoic acid EPA and endocannabinoid eCB with therapeutic enhancement for brain protection and repair.
   Food enrichment with DHA and other long-chain ω-3, such as EPA, has shown beneficial effects on learning and memory, neuroinflammatory processes, synaptic plasticity and neurogenesis.
   ARA, DHA and EPA are precursors to a diverse repertoire of bioactive lipid mediators, including endocannabinoids.
   The endocannabinoid system includes cannabinoid receptors, their endogenous ligands, endocannabinoids, and their biosynthetic and degrading enzymes. Anandamide (AEA) and 2-arachidonoylglycerol (2-AG) are the most studied endocannabinoids and are both derived from phospholipid-bound AA.
2. There is a complex interaction between ω-3, ω-6 and the endocannabinoid system with therapeutic potential for brain protection and repair.
   For example:
      long-term dietary supplementation of DHA and EPA reduces levels of AEA and 2-AG, with reciprocal increases in levels of the similar DHA- and EPA-derived endocannabinoid-like molecules [Dyall S C 2017].
      The ω-3, particularly docosahexaenoic acid, during postnatal brain development shape synaptic plasticity in the hippocampus, which affects long-term memory and cognitive disorders. Activity-dependent plasticity at excitatory and inhibitory synapses in the CA1 region of the hippocampus mediated by endocannabinoids produced by the post-synapse is impaired by a deficiency of ω-3 [Thomazeau A et al. 2017].
      Long-term exposure to a diet lacking in ω-3 reduces the level of DHA in the brain and alters the signaling pathway of cannabinoid receptor in the mood and anxiety control structures of the prefrontal cortex and in the hypothalamus. Consequently, experimental data in mice suggest that behavioral changes related to a dietary deficiency of ω-3 are due to an alteration of the endocannabinoid system in specific areas of the brain [Lafourcade M et al. 2011, Larrieu T et al. 2012].
3. Emerging evidence suggests a complex interaction between the endocannabinoid system, ω-3 fatty acids, ω-6 fatty acids, and the immune system in promoting brain self-repair. EPA and DHA have distinct effects in the neural stem cell (NSC) fate regulation that are mediated by endocannabinoid signaling pathways:
   EPA, but not DHA, significantly increases NSC proliferation compared to controls; an effect associated with increased levels of the endocannabinoid 2-arachinoylglycerol (2-AG), p-p38MAPK e IL-1β.
   DHA promotes neuronal differentiation of NSCs. DHA is metabolised to synaptamide in cultured NSCs. Synaptamide potently induces neuronal differentiation of NSCs. Synaptamide-induced neuronal differentiation is mediated by activation of protein kinase A (PKA/CREB) [Rashid M A et al. 2013].
   The DHA effects are mediated by alternative signaling pathways such as some hippocampal endocannabinoid/endovanilloid receptor subtypes with increased spatial memory induced by DHA integration [Pan J-P et al. 2011, Dyall S C 2015].
4. Interconnections and interactions between PPARs, DHA and βCP PPARs (peroxisome proliferator-activated receptors) are a family of nuclear receptors including PPAR-α, PPAR-γ e PPAR-β/δ, which act as transcription factors to regulate the expression of a plethora of target genes involved in metabolism, immune response, cell differentiation, and a variety of other cellular changes and adaptive responses.
   PPARs have been recognized as sensitive receptors for a variety of endogenous lipids (such as monounsaturated and polyunsaturated fatty acids) and natural exogenous compounds. For example, fatty acids ω-3 (EPA and DHA) activate PPAR-γ either through direct binding [Grygiel-Górniak B 2014] or through the PI3K (phosphatidylinositol-3-kinase) pathway mediated by GPR120 (G-protein coupled receptor 120) [Hasan A V et al. 2015].
   PPARs are activated by a large number of both endogenous and exogenous lipid molecules, including phyto- and endocannabinoids, as well as endocannabinoid-like compounds. In this perspective, they can be considered an extension of the endocannabinoid system. As previously reported, βCP is ligand and inducer of overexpression of PPARs receptors [Wahli W 2008].
   In addition to being activated directly by cannabinoids, PPARs are also indirectly modulated by receptors and enzymes that regulate endocannabinoid activity and metabolism, and conversely, the expression of these receptors and enzymes can be regulated by PPARs [Iannotti F A and Vitale R M 2021].

### B. Interactions Between βBA, βCP, DHA

There is an effective neuroprotective interaction between βBA, βCP and DHA.

Indeed, βBA:
Has an anti-inflammatory action similar to NSAIDs (Non-Steroidal Anti-Inflammatory Drugs) and steroids [Dahmen U et al. 2001].
It is a direct inhibitor of 5-LOX (5-lipoxygenase), a key enzyme in the leukotrienes biosynthesis from arachidonic acid [Safayhi H et al. 1992, Gilbert N C et al. 2020].
It is an inhibitor of cyclooxygenase-1 (COX-1) and COX-2. It is an inhibitor of prostaglandins PGE₂ by inhibiting mPGES-1 (prostaglandin-E-synthase-1 microsomal). Indeed, many evidences show a perinuclear and cytoplasmic colocalization of COX-2 and mPGES-1 [Siemoneit U et al. 2010, Verhoff M et al. 2014] with neuroprotective effects both in vivo and in vitro in models of stroke, Alzheimer's disease, Parkinson's disease, multiple sclerosis, amyotrophic lateral sclerosis, epilepsy, schizophrenia, Huntington's disease [Yagami T et al. 2015].
It has an anti-inflammatory action, maintenance of neuronal integrity, myelin stability, myelin regeneration and reperfusion against ischemic insult, through activation of the gene transcription factors Nrf2 (Nuclear related factor 2) and HO-1 (heme oxygenase-1) [Pareek T K et al. 2011, Ding Y et al. 2014]. This same mechanism can be exploited to protect against microcirculatory damage present in the Alzheimer's disease-amyotrophic lateral sclerosis-parkinsonism-dementia complex [Buée L et al. 1997, Roy N K et al. 2019].
It prevents tight junctions destruction induced by oxidative and inflammatory stimuli; counteracts H₂O₂-induced ROS generation [Ammon H P 2010, Liang Y H et al. 2010, Catanzaro D et al. 2015].
It modulates the immune response: inhibits the NF-kB activation and reduces pro-inflammatory cytokines TNFα, IL-1, IL-2, IL-4, IL-6, IFNγ; suppresses the classical complement pathway with the conversion of C3 into C3a and C3b inhibition. This improves learning and memory [Marefati N et al. 2020].
It regulates the injured peripheral nerves repair by promoting the Schwann cells proliferation [Jiang X et al. 2020].
It increases the BDNF expression [Asadi E et al. 2019].
It helps to maintain the ions homeostasis such as iron, copper, magnesium, zinc and calcium in the brain and thus to counteract the Alzheimer's advancement [Wang L et al. 2020]. For example, it suppresses the intracellular Ca⁺⁺ mobilization [Siemoneit U et al. 2017] and thus favorably affects glutamatergic transmission and memory storage; it is neuroprotective against glutamate damage by inhibiting apoptotic neuronal death [Rajabian A et al. 2019].
It has antiamyloidogenic action [Rajabian A et al. 2019] because it inhibits the β-amyloid and Tau proteins deposition [Haghaei H et al. 2020], reduces β-amyloid levels and improves cognitive impairment [Wei C et al. 2020].

### C. Interactions Between FE, βCP, βBA, DHA

### 1. M-Opioid Receptors and CB₂ Receptors.

The μ-opioid agonists and CB₂ agonists act synergistically to inhibit chronic pain by reducing unwanted opioid-induced side effects. The opioid sparing effect of CB₂ receptor agonism strongly supports the advancement of a combined CB2-agonist μ-opioid pain therapy [Grenald S A et al. 2017] and can provide new strategies for therapies in addicted subjects [Befort K 2015].
The association of myrrh (conteining FE) and β-caryophyllene was found to be effective on chronic neuropathic pain [Fotio Y et al. 2019].
Boswellia resin causes behavioral, antidepressant and anxiolytic effects in mice [Moussaieff A and Mechoulam R 2009] with predictable synergistic action with FE.
The combination of FE+βCP can increase the clinical efficacy of opioids, improve tolerance and reduce opioid dependence. Indeed, stimulation of the CB₂ receptor attenuates the microglial inflammation induced by morphine [Gessi S et al 2016] by inhibiting microglia activity, thus may be potential target for increasing the opioids clinical efficacy [Merighi S et al. 2012].

### 2. M-Opioid Receptors, CB₂ and 3BA Receptors in Psychic Disorders of Sexuality in Women and Men and in Infertility.

βCP has aphrodisiac activity, demonstrated in women (increase in testosterone levels in saliva after terpene nasal inhalation) [Tarumi W and Shinohara K 2020];
βCP promotes fertility [Maccarrone M 2008];
βBA on mouse model demonstrated efficacy on sperm viability, spermatogenesis and fertility [Tohamy H G et al. 2021];
FE is effective on anxiety and depression.

Composition or association of compounds object of this invention contemplating sublingual administration, of the same efficacy as the olfactory route, of βCP, FE, βBA, DHA, HA₄ all together with their activity on the whole nervous system particularly on the CB₂, endocannabinoid system, opioid receptor μ1, system, monoaminergic neurotransmission, lipid metabolism, can be exploited to solve the psychological problems of sexuality in women (sexual desire/interest disorders, sexual arousal disorders, orgasm disorders, vaginismus, dyspareunia, tubal cilia hypomotility infertility, postpartum depression) and men (reduced libido, eiaculatio precox, erectile dysfunction, impotence, sperm hypomotility infertility).

### 3. M-Opioid Receptors and Endogenous DHA.

Feeding disorders with behavioral disorders. In humans, children of obese mothers and children of lean mothers due to nutrient or calorie deficiency are at increased risk for certain neurodevelopmental disorders, including attention deficit/hyperactivity disorder, schizophrenia, and social and humor disorders. In addition, these offspring exhibit alterations in the mesocorticolimbic genes expression that regulate dopamine and opioid function, particularly μ-opioid receptors, thus indicating that these brain regions and neurotransmitter systems are vulnerable to gestational insults [Thanos P K et al. 2018].

In murine experiments, morphine reduced striatal DHA content and this was reversed by supplemental ω-3 [Hakimian J et al. 2017].

It is object of the present invention the composition or association of compounds with βCP-FE-βBA-DHA-HA₄ for their perfect interconnection and seamless integration resulting in further synergistic enhancement on the prevention and repair of neurological damage and pathophysiological neuronal function even at advanced tissue damage when all therapies to date, including immunotherapy or stem cell therapy itself, have failed. Indeed, the compound can make even immunotherapy effective and help stem cell action. This is the resilience effect of the composition or combination of compounds.

It is object of the present invention the composition or association of compounds with βCP-FE-βBA-DHA-HA₄ for use in the treatment of mental disorders of male and female sexuality and infertility from hypospermia and spermatic and ciliary tubal motility deficits.

D. βCP-FE-@BA-DHA-HA₄ for the Prevention and Treatment of Neurological Damage from Neurotropic Viruses

### HIV and SARS-COV-2 Diseases are Paradigmatic Examples

HIV. HIV entry into the central nervous system (CNS) is known to occur in the first week (or weeks) after infection. Today, combined antiretroviral therapy is the standard treatment for all people with HIV; although it has improved the quality of life of people living with HIV, it cannot eliminate the latent reservoir of the virus. Therefore, HIV/AIDS has transformed from a fatal disease into a chronic disease that requires lifelong care.

Despite significant viral load suppression, it has been observed that at least half of patients receiving combination antiretroviral therapy have HIV-associated neurocognitive disorders, which have been related to HIV-1 infection and replication in the CNS.

Entered into the brain, HIV-1 can generate an inflammatory environment by causing the release of viral proteins (such as Tat and gp120) and cellular products (such as proinflammatory cytokines, e.g. TNF-α, IL-8, IL-6 and IL-1β). Infection involves all components: microglia, perivascular macrophages, astrocytes, oligodendrocytes and neurons themselves [Rojas-Celis V et al. 2019].

COVID-19. Growing reports have shown that SARS-COV-2 infection involves the CNS and the peripheral nervous system (PNS). It is likely that SARS-COV-2 uses the immune system cells to spread throughout the body and cross the blood brain barrier (BEE) in a HIV very similar process. In addition, it can enter the brain via the optic and olfactory nerve channels and through vascular endothelial cells.

The SARS-COV-2 epidemic can cause various types of neurological damage, including Guillain-Barré syndrome axonal variant, ischemic stroke with the formation of fatal microthrombi, seizures, even onset of encephalitis, long-term neurological sequelae [Wang et al. 2020].

Common to HIV-AIDS and SARS-COV-2/COVID-19 is the systemic increase in inflammatory mediators, now called a “cytokine storm”, which could explain multi-organ damage and their effects on the CNS and PNS. Indeed, the release of large numbers of proinflammatory cytokines increases vascular permeability, abnormal blood clotting and multi-organ failure. These cytokines may also play a role in increasing microvascular permeability in the CNS, facilitating the entry into the brain of HIV and SARS-COV-2 through BEE. The “cytokine storm” can also promote the formation of microthrombi by activating the coagulation system.

βCP is a candidate to target the triad of infection, immunity and inflammation in SARS-COV-2/COVID-19 [Jha N K et al. 2021] and in HIV/AIDS. In fact, βCP has therapeutic effects both in HIV/AIDS and in COVID-19 since:
It has antiviral activity on SARS-COV-2 [Narkhede R R et al. 2020] and HIV [Zubair M S et al. 2021].
It modulates the immune-inflammatory response with activation of the type 2 cannabinoid receptors (CB₂R), which modulates numerous signaling pathways and nuclear receptors in particular the receptors activated by the proliferation of peroxisomes (PPAR) [Jha N K et al. 2021](see above).
Suppresses or reduces mechanical allodynia in HIV patients with neuropathic pain [Aly E et al. 2019, Aly E and Masocha W 2021].

The βBA has a modest direct anti-SARS-COV-2 [Roy A and Menoin T 2021], but has a more relevant anti-inflammatory action, inhibiting secretion of pro-inflammatory cytokines such as TNFα, IL-1, IL-6, IL-12, IL-18, IFN-γ [Cavaillon J M 2001, Gomaa A et al. 2021]. It also has antioxidant and neuroprotective effects on synaptic plasticity [Marefati N et al. 2020].

The composition or association of compounds of the present invention includes not only βCP and βBA but also:
the addition of FE which acts on the endogenous morphine system and therefore on pain and cenesthesia;
the addition of DHA which acts on the lipid metabolism of ω-3 fatty acids and arachidonic acid and therefore on cellular homeostasis and tissue inflammation;
the addition of HA₄ which supports the recovery of neuronal damage.

All of this constitutes a real step forward in the treatment of these devastating pandemic diseases.

It is, therefore, an object of the present invention the composition or association of compounds with DHA-HA₄-βCP-FE-βBA for use in prevention, treatment, as adjuvant, as integrative:
in acute and chronic infectious virus diseases, in particular with neurological involvement of the CNS and PNS (cytomegalovirus encephalitis, chicken pox, herpes zoster and simplex virus; myelitis, polyradiculopathies);
in HIV disease (acute, subacute and chronic encephalitis; HIV-dementia complex, polyradiculopathies);
in SARS-COV-2/COVID-19 disease (hyposmia and anosmia; cognitive deficits; onset or aggravation of Alzheimer's, Parkinson's, multiple sclerosis; COVID 19-dementia complex, their complications and long-term sequelae).

### IV THE COMPOSITION OR ASSOCIATION OF COMPOUNDS AND ITS PREPARATION

### Material

The following material is provided for the present invention:
docosahexaenoic acid DHA (C22:6 ω-3—C₂₂H₃₂O₂—MW 328.488—Hydrogen bond donor count 1—Hydrogen bond acceptor count 2). As DHA triacylglycerol available on the market;
hyaluronic acid HA₄ tetrasaccharide (C₂₈H₄₄N₂O₂₃—MW˜776 Da—Hydrogen bond donor count 14—Hydrogen bond acceptor count 23) sodium salt. This product is available on the market;
β-caryophyllene βCP (C₁₅H₂₄—MW 204.35—Hydrogen bond donor count 0—Hydrogen bond acceptor 0). Available on the market;
furanoeudesma-1,3-diene FE (C₁₅H₁₈O—MW 214.30—Hydrogen bond donor count 0—Hydrogen bond acceptor count 1). Available on the market;
ß-boswellic acid βBA (C₃₀H₄₈O₃—MW 456.7—Hydrogen bond donor count 1—Hydrogen bond acceptor count 3; “Lipinski's rule of five” 1 [Vijayarani K R et al. 2020]). Available on the market.

### Method

The method according to the present invention provides:
1. Obtaining three nanoparticle compounds HA₄-βCP, HA₄-FE, HA₄-βBA by exploiting the bonds that are formed between the hyaluronic acid HA₄ tetrasaccharide with the two sesquiterpenes β-caryophyllene βCP and furanoeudesma-1,3-diene FE, and with the pentacyclic triterpene β-boswellic acid βBA, subjected to an electrostatic field system [Kao Y-H et al. 2012].
2. Obtaining an emulsion by mixing HA₄-βCP, HA₄-FE, HA₄-βBA compound with the ω-3 DHA by sonification.
3. Possible sweetening and aromatization of the emulsion for a good palatability and digestibility.
4. Product formulation.

The preparation of compositions with DHA-HA₄-βCP-FE-βBA takes into account:
the final DHA concentration which in the desired product must be at least 400 mg/ml.
the final HA₄ concentration which in the desired product must be 80 mg/ml.
the final βCP concentration which in the desired product must be 20 mg/ml.
the final FE concentration which in the desired product must be 20 mg/ml.
the molecular weight of HA₄ 776 Da and the molecular weight of βBA 457 Da with a ratio HA₄: βBA=1.69.
the final βBA concentration which in the desired product must be 40 mg/ml.
the expected therapeutic dose in humans of DHA, HA₄, βCP, FE, βBA ingested sublingually: of at least 400 mg/ml for DHA; of 40-80 mg/day for HA₄, of 10-20 mg/day for βCP and for FE; of 20-40 mg/day for βBA.

These proportions are indicative and may vary according to the pathophysiological mechanisms involved in the various diseases, with prevalent damage to HA₄, or βCP, or FE, or βBA.

In practice, with the five components contemplated in the present invention, various compositions and associations of compounds are provided. For example:

A. Composition of the 5 Active Principles Formed by the Association of ω-3 DHA with 3 Compounds of HA Nanoparticles Aggregated with an Electrostatic Field System to βCP, FE and 3BA Respectively.

Association components:
ω-3 DHA: ml 400
+—Compound a) HA₄/βCP:
   βCP 20 g in 100 ml H₂O (ml 333 hydroalcoholic solution with 70% ethanol)
   HA₄ 20 ml in 100 ml H₂O
+—Compound b) HA₄/FE:
   FE 20 g in 100 ml H₂O (ml 333 hydroalcoholic solution with 70% ethanol)
   HA₄ 20 ml in 100 ml H₂O
+—Compound c) HA₄/βBA:
   βBA 40 g in 100 ml H₂O (ml 333 hydroalcoholic solution with 70% ethanol)
   HA₄ 40 ml in 100 ml H₂O
      B. Composition of 4 Active Principles Formed by the Association of ω-3 DHA with 2 Compounds of HA₄ Nanoparticles Aggregated with an Electrostatic Field System to βCP and FE.

Association components:
ω-3 DHA: ml 600
+Compound a) HA₄/βCP:
   HA₄ 40 ml in 100 ml H₂O
   βCP 20 g in 100 ml H₂O (ml 333 hydroalcoholic solution with 70% ethanol)
+Compound b) HA₄/FE:
   HA₄ 40 ml in 100 ml H₂O
   FE 20 g in 100 ml H₂O (ml 333 hydroalcoholic solution with 70% ethanol)
      C. Composition of 3 Active Principles Formed by the Association of ω-3 DHA with a Compound of HA₄ Nanoparticles Aggregated with an Electrostatic Field System to βCP.

### Components:

| | |
|---|---|
| ω-3 DHA: | ml 800 |
| HA₄ | ml 80/1 in 100 ml H₂O |
| βCP | g 20/1 in 100 ml H₂O |
| | (ml 333 hydroalcoholic |
| | solution with 70% ethanol). |

D. Composition of 3 Active Principles Formed by the Association of ω-3 DHA with a Compound of HA₄ Nanoparticles Aggregated with an Electrostatic Field System to FE.

### Components:

| | |
|---|---|
| ω-3 DHA | ml 800 |
| HA₄ | ml 80/1 in 100 ml H₂O |
| FE | g 20/1 in 100 ml H₂O |
| | (ml 333 hydroalcoholic |
| | solution with 70% ethanol). |

E. Composition of 3 Active Principles Formed by the Association of ω-3 DHA with a Compound of HA₄ Nanoparticles Aggregated with an Electrostatic Field System to 3BA.

### Components:

| | |
|---|---|
| ω-3 DHA | ml 800 |
| HA₄ | ml 80/1 in 100 ml H₂O |
| βBA | g 40/1 in 100 ml H₂O |
| | (ml 333 hydroalcoholic |
| | solution with 70% ethanol). |

F. Composition of 2 Active Principles Formed by the Association of ω-3 DHA with HA₄ Nanoparticles, or with βCP, or with FE, or with βBA.

### Components:

| | |
|---|---|
| ω-3 DHA | ml 900 |
| + | |
| HA₄ | ml 80/1 in 100 ml H₂O |
| or | |
| βCP | 20 g in 100 ml H₂O |
| | (ml 333 hydroalcoholic |
| | solution with 70% ethanol) |
| or | |
| FE | g 20/1 in 100 ml H₂O |
| | (ml 333 hydroalcoholic |
| | solution with 70% ethanol) |
| or | |
| βBA | g 40/1 in 100 ml H₂O |
| | (ml 333 hydroalcoholic |
| | solution with 70% ethanol). |

The technique here referred to is only as an example and refers to the type A association with ω-3 DHA+all the three compounds and it also applies to the other associations in B, C, D, E, F.

I Time. Obtaining Four Solutions Containing Each:
a) g 20 of FE dissolved in 333 ml of hydroalcoholic solution (ethanol 70%, water 30%);
b) g 20 of βCP dissolved in 333 ml of hydroalcoholic solution (ethanol 70%, water 30%);
c) g 40 of βBA in 333 ml of hydroalcoholic solution (ethanol 70%, water 30%);
d) g 80 of HA₄ in 100 ml of distilled water.

Mixing of the three solutions (a), (b), (c) in a rotating magnetic field with intensity ranging from 100 to 300 mT (milliTesla), preferably between 150 and 200 mT. Temperature between 6° and 75° C., preferably around 75° C. Operation carried out with an average time of 90 minutes, oscillating between 60 and 120 minutes.

Mixing HA₄ with distilled water by sonication (20 kHz at 30% of 130 Watt, 3 times for 5 seconds).

II Time. Ethanol Vaporization.

The three solutions (a), (b), (c) are separately subjected to a rotating magnetic field for a variable time of 60-120 min with field strengths between 30 and 100 mT (milliTesla), preferably between 50 and 75 mT at a temperature of 85±5° C. Since ethanol has its boiling point at 78.4° C., it is completely removed by evaporation.

Three aqueous solutions of 100 ml with 20 mg/ml of βCP; 100 ml with 20 mg/ml of FE; 100 ml with 40 mg/ml of βBA, respectively, are obtained.

The three solutions are mixed with the three HA₄: solutions: each mixture by sonication at 20 kHz at 30% of 130 Watt, 3 times for 5 seconds.

III Time. Obtaining Three Compounds of HA₄ Nanoparticles and Aggregates HA₄/βCP, HA₄/FE, HA₄/βBA with an Electrostatic Field System.

HA₄ nanoparticles are produced using a well-known method of preparing biopolymer nanoparticles with an electrostatic field system [Kao Y-H et al. 2012, Sun Q et al. 2013]. In this method, HA₄ nanoparticles are well dispersed in solution and show a narrow range of size less than 1 nm. The negatively charged HA₄ enhances water solubility, contributes to the stable aggregation of the three terpenes to these biopolymeric nanoparticles, improves bioavailability by increasing degree and speed to pass into the systemic circulation.

Mixing of the three new solutions (HA₄+βCP; HA₄+FE; HA₄+βBA) by sonication (sonication at 20 kHz at 30% of 130 Watt, 3 times for 5 seconds).

Each new solution is subjected to a high-intensity electrostatic field (2.5 kV/cm) and temperature of 25° C. for 60 min.

Three compounds consisting of well-dispersed HA₄ nanoparticles and HA₄/βCP, HA₄/FE, HA₄/βBA aggregates of homogeneous size around 1 nm are obtained, respectively.

IV Time. Mixing of the Three Compounds with DHA.

The three compounds HA₄/βCP, HA₄/FE, HA₄/βBA are mixed with 400 ml of DHA by sonication to obtain a homogeneous and stable emulsion (sonication at 20 kHz at 30% of 130 Watt, 3 times for 5 seconds).

A 1,000 ml compound is obtained containing: 80 mg/ml of HA₄; 20 mg/ml of βCP; 20 mg/ml of FE, 40 mg/ml of βBA, i.e., the amount that corresponds to the daily doses considered therapeutic according to the present invention.

### Conclusion

The present invention provides for the use of five elements (DHA, HA₄, βCP, FE, βBA) quite known in their actions that are complementary, but the invention is based both on the choice of the single components and above all on their composition or association.

Furthermore, with the combined method of the rotating magnetic field and the electrostatic field, a compound with a higher density of nanoparticles is obtained in an electrostatically stable solution at room temperature.

Another advantage is that the preparation does not need special nanoengineering techniques, which require high technology and high production costs.

Another advantage is constituted by the fact that the electrostatic field system allows, much more effectively than has been attempted so far, to eliminate the need to build nanocapsules, nanogels, nanoparticles of bioactive compounds and problems of overcoming barriers.

Another advantage is that this preparation due to the size of the individual compounds in the composition or association has particles less than 1 nm and easily crosses the sublingual and capillary mucosal barrier.

Another advantage is that the electrostatic field system allows bonds between HA₄ and βBA, HA₄ and FE, HA₄ and βCP with “weak” but sufficiently stable non-covalent interactions with the formation of HA₄-terpene aggregates, nanoparticles which have the property of easily crossing both the sublingual mucosal barrier and the blood brain barrier.

Another advantage is that despite being conceived as a nutraceutical product to be administered sublingually, the composition or association of compounds crosses the skin, gastro-intestinal mucosal, pulmonary barriers and can reach the central nervous system (brain and spinal cord) transnasally too.

Another advantage is that hyaluronic acid not only acts as a support and carrier of another drug but is itself the therapy fundamental element. Conversely, the most significant literature data concern the delivery of nanoconjugated drugs with a large hyaluronic acid, the smallest of 6-8 monosaccharides (HA₆₋₈). But these oligomers are captured by membrane receptors, such as CD44 and RHAMM, and then endocytized, without carrying out that beneficial activity in the extracellular matrix and in synergy, which is only possible with the preparation of this invention.

Another advantage is that it transports the drug inside the cell, and this further enhances the effect of the transported drug, regardless of the fact that DHA, HA₄ and the three terpenes βCP, FE and βBA are synergistic.

### The Advantages of the Sublingual Formulation

It is the first time that the substances contemplated in this formulation, considered both individually and together, are administered sublingually (i.e. in the area under the tongue). Method we cited in a recent paper [Morgese M G et al. 2021].

The sublingual route compared to the gastrointestinal route offers substantial advantages in Alzheimer's disease and in general in the Nervous System diseases.

In fact, the sublingual route allows:
Qualifying the product as a nutraceutical as it essentially is, while the use of the olfactory or pulmonary inhalation route would qualify it as a drug and would require times for authorization that would penalize many people who can benefit from it in the meantime. But we also claim the via transnasal and pulmonary routes product administration invention.
Reducing the dosage and side effects. In fact, the sublingual route undergoes a much less gastrointestinal mucosa barrier thus with a minimal pre-blood loss. In practice, βBA is reduced up to 40 times when passing from the intestinal mucosa to the blood [Gerbeth K et al. 2013] while it is reduced by 45% sublingually, according to our experiments. The better absorption is due to the fact that the sublingual lining mucosa is not keratinized; is very distensible compared to the intestinal mucosa; has a thinner absorption surface and greater paracellular permeability; allows greater fat solubility with greater absorption of substances with high in lipids solubility and poor in water solubility.
Having a highly vascularized sublingual area with a tributary venous circulation of the superior vena cava. Thus, the drug quickly reaches the systemic circulation, and thus the brain, almost like an intravenous injection.
Getting the drug in higher concentration and unmodified to the target organ, i.e., the brain, on first passage through the systemic circulation while avoiding hepatic metabolism where it undergoes significant metabolism [Krüger P et al. 2009].
It is likely that the effectiveness of the sublingual pathway is comparable to the “olfactory pathway of terpenes” [Bevilacqua M, Masson Ed. 2005: 144-149] and as an alternative to it. In fact, a remarkable variety of enzymes are present both at the blood-blood-brain barrier level and in the brain tissue [Pavan B et al. 2008] where the composition or association of compounds can undergo intense biotransformation. This probably explains the reduction in blood levels, which we recorded in mice in the first minutes after the administration of βBA (FIG. 2).
   In this regard, it should be noted that:
      β-caryophyllene, although there are no safety problems, however long-term and high-dose via os administration induced hepatocyte hypertrophy in the mouse [Bastaki M et al 2020];
      hyaluronic acid would be captured largely in the liver before reaching the brain, with a reduction in concentration, according to our calculations, by thirteen times.
Absorbing anyway DHA triglycerides, which undergo digestion due to the of salivary lipase presence, releasing DHA. [βCP, FE and DHA are totally absorbed through the sublingual mucosa like via os administration]
Self-administering the preparation comfortably with better compliance in patients who have difficulty swallowing.
Having a product that can be sweetened and flavored so that the emulsion has a pleasant taste, formulated in a way suitable to be taken in this way.
The sublingual formulation may be more advantageous than oral use also because it is cheaper (such as Boswellia serrata chewing gum [Gomaa A A et al. 2021]).

Practically, the preparation of the present invention is a real step forward both because it combines five substances that are synergistic in counteracting the development of Alzheimer's disease and other neurological diseases and because of the significant dose saving of the components with result of greater efficacy and without side effects.

### V EXPERIMENTAL METHODOLOGY

The trials were performed at the Department of Clinical and Experimental Medicine of the University of Foggia according to the following rationale.
1. In the present mouse model of beta-amyloid toxicity, using validated tests in mice, we evaluated the behaviors:
   a. antidepressant: Tail Suspension Test (TST); Forced Swimming Test (FST); Splash Test (ST) with latency time (sec) and self-care (sec) measurement.
   b. antianxiety: Open Field Test with measurement of distance traveled (m), time spent in the wall (sec), time spent in the center (sec), center entries (n); Freezing Test (sec).
   c. on memory, learning, coordination of motor skills, fear conditioning: Rotarod Test with measure of Latency to Fall, repeated after training to evaluate motor learning.
2. We tested the protective effects with the following procedure:
   a. individually: βCP, FE, βBA, DHA and HA₄;
   b. in association with pairs: βCP+HA₄, FE+HA₄, βBA+HA₄, FE+βCP, βBA+FE, βBA+βCP.
   c. in association, all four together: βCP+FE+βBA+HA₄
   d. versus control (SHAM),
   e. using the mouse model of an Aβ-induced depressive phenotype.
3. These behavioral tests were integrated with serotonin, dopamine, noradrenaline and their metabolites prefrontal cortex and hippocampus measurement.
4. Furthermore, we evaluated the cortical and hippocampal glutamate (GLU) levels in mice treated with AP and subsequently we tested the ability of βCP, FE, βBA and HA₄, to modulate this neurochemical parameter.

This is because alterations in glutamatergic function have also been postulated as an alternative to the monoaminergic hypothesis of depression [Sanacora G et al. 2012]; moreover, together with neuroinflammation, AD is also characterized by excitotoxic levels of extracellular glutamate [Hiruma H et al. 2003, Kopeikina K J et al. 2012].

5. On the other hand, we have previously found that intracerebroventricular injection of the peptide was accompanied by an increase in kynurenine (KYN) levels [Morgese M G et al. 2021]. This molecule is produced from tryptophan after enzymatic bioconversion by indolamine 2, 3-dioxygenase (IDO) enzymes.

The metabolic shift from tryptophan metabolism to KYN and its derivatives, such as kynurenic or quinolinic acids, instead of 5-HT has been proposed as another possible biological mechanism under evaluation to explain a depressive state [Oxenkrug G 2013].

Interestingly, a crucial crosstalk between the KYN pathway and glutamatergic function has been described [Schwarcz R 2016]. Therefore, we also investigated the role played by βCP, FE, βBA and HA₄ in regulating the interconnection between these biological substrates in the animal model treated with Aβ.

6. Dystrophic microglia in the human brain is associated with neurodegenerative Alzheimer's disease, Lewy body dementia, Huntington's disease, multiple sclerosis, Down syndrome [Xue Q S, Streit W J 2011], limbic predominantly age-related TDP-43 encephalopathy and unhealthy aging [Bachstetter A D et al. 2015, Shahidehpoura R K et al 2021]. Glial cells play a crucial role in maintaining GLU homeostasis, along with regulating the pro-inflammatory biomarkers production after toxic brain insults, such as extra-physiological levels of Aβ. Indeed, astrogliosis and activation of microglia have been described in vivo after intracerebroventricular injection of Aβ by our group and by other researchers [Bove M et al. 2018]. Therefore, to understand the purported neuroprotective mechanism of action of βCP, βBA, FE and HA₄, we quantified biological biomarkers associated with glial activation such as glial fibrillar acid protein (GFAP), for astrocytes [Yang Z and Wang K W 2015], and CD11b, a marker of activated macrophages and microglia [Roy A et al. 2008].

7. Again, it has been shown that βBA could exert its beneficial effects with the BDNF expression and suppressing the gene expression regulated by the nuclear factor kappa-B (NF-kB) [Takada Y et al. 2006]. NF-kB is a heterodimeric transcription factor that plays a crucial role in orchestrating the immune response and neuroinflammation. This transcription factor is activated by prostanoids and proinflammatory cytokines which consequently activate NF-kB generating a vicious circle [Orban Z et al. 2000]. Therefore, the role played by NF-kB in inducing the Aβ-depressive phenotype and, in turn, the βCP, FE, βBA and HA₄ on these mechanisms has been studied.

8. Again, since soluble amyloid-8 oligomers alter synaptic transmission, in particular:
induce caspase-dependent loss of two synaptic proteins, PSD-95 and synaptophysin, via NMDA receptors [Liu J et al. 2010];
induce disorders in the PSD-95 (Post-Synaptic Density-95) expression and affect long-term depression (LTD) [Dore K and Malinow R 2021];
act on the neurotransmitters release by destroying the interaction between synaptophysin and VAMP2 [Russell C L et al. 2012];
induce a dysregulation of the calcium/calmodulin-dependent protein kinase II CaMKII [Ghosh A and Giese KP 2015].

The βCP, FE, βBA and HA₄ influence on prefrontal and hippocampal synaptic plasticity was evaluated using anti-PSD-95, anti-synaptophysin, anti-CaMKII antibodies.

9. Finally, experiments are ongoing with ω-3 DHA associated with pairs: DHA+βCP; DHA+βBA; DHA+FE; DHA+HA₄; all together: DHA+βCP+βBA+FE+HA₄, but, since treatment with ω-3 takes a few weeks, the results will be collected in due course.

Moreover, the results of the experiments at the same Department of the University of Foggia on the ability of ω-3 to prevent oxidative stress induced by Aβ have recently been published [Morgese M G et al. 2021] and these are added to those concerning the preventive antidepressant action of ω-3 in mice treated with Aβ, previously published [Colaianna M et al. 2010; Morgese M G et al. 2017, 2018, 2020; Bove et al. 2018].

In Summary, the Prospectus of the Investigations Carried Out in this Study
a. Behavioral tests: TST; FST; Rotarod with Latency measurement, repeated after training to evaluate motor learning; Open field, with distance traveled measurement, Time spent in the wall, Center entries; Freezing. Tests performed with individual compounds, in pairs, all together according to the scheme in No. 2.
b. Behavioral tests integrated with the measurement of serotonin, dopamine, noradrenaline and their metabolites in the prefrontal cortex (CPF) and in the hippocampus (IPP). Tests performed with individual compounds, in pairs, all together according to the scheme in No. 2.
c. CPF and IPP levels of kynurenine (KYN), kynurenic acid (KYNA) and quinolinic acid (QUIN) enzyme indolamine 2, 3-dioxygenase (IDO) in mice treated with AP, and after 7 days, separately, with βCP, βBA, FE, HA₄; and then with βCP+βBA+FE+HA₄ together.
d. Levels in CPF and IPP of glutamates in mice treated with AP; in mice treated with AP and after 7 days, separately, with βCP, βBA, FE, HA₄; and then with § CP+βBA+FE+HA₄ together.
e. Levels in CPF and IPP of glial fibrillary activation biomarkers GFAP and astrocytic CD11b in mice treated with Aβ; in mice treated with AP and after 7 days, separately, with βCP, βBA, FE, HA₄ and then with § CP+βBA+FE+HA₄ together.
f. CPF and IPP measurement of nuclear factor NF-kB in mice treated with Aβ; in mice treated with AP and after 7 days, separately, with βCP, βBA, FE, HA₄ and then with βCP+βBA+FE+HA₄ together.
g. CPF and IPP measurement of anti-PSD-95, anti-synaptophysin, anti-CaMKII antibodies in mice treated with AP; in mice treated with AP and after 7 days, separately, with βCP, βBA, HA₄ and then with βCP+βBA+FE+HA₄ together.

### VI MATERIAL AND METHOD

### Substances

hyaluronic acid HA₄ tetrasaccharide was purchased by the Merck Life Science company (Milan)
β-boswellic acid βBA was purchased by the Merck company.
β-caryophyllene βCP was purchased from Cayman Chemical Company MI, USA (Vinci-Biochem distributor, Florence Italy).
furanoeudesma-1,3-diene FE was purchased from the firm Merck (Germany), distributor Sigma.
docosahexaenoic acid DHA as DHA triglyceride was purchased by the Fermentalg company (33500 Libourne France).

### Animals

The experiments were conducted using a group of 8-10 week-old male C57/B16 mice and then other groups of 10-12 week-old male C57/B16 mice as needed for subsequent experiments (Envigo, San Pietro al Natisone, Italy). They were housed at constant room temperature (22±1° C.) and relative humidity (55±5%) constants, under a 12h light/dark cycle. Water and food were available ad libitum. Procedures involving animals and their care were conducted in conformity with the institutional guidelines of the Italian Ministry of Health (DL 26/2014), the Guide for the Care and Use of Laboratory Animals: Eighth Edition, the Guide for the Care and Use of Mammals in Neuroscience and Behavioral Research (National Research Council, 2004), the Directive 2010/63/EU of the European Parliament and of the Council of 22 Sep. 2010 on the protection of animals used for scientific purposes, in accordance with ARRIVE guidelines. Animal welfare was daily monitored through the experimental period and all efforts were made to minimize the number of mice used and their suffering. The experimental protocol was approved by the Italian Ministry of Health (approval number 665/2019-PR, protocol n. B2EF8.23).

### Surgery and Amyloid Beta Administration

The Aβ₁₋₄₂, obtained from Tocris (Bristol, UK), was dissolved in sterile double-distilled pyrogenic-free water, as vehicle, to obtain a final concentration 4 μM [Colaianna M et al. 2010]. Mice were anesthetized with a solution (0.85 in ml/kg, i.p.), containing ketamine (Sigma Aldrich, Milan, Italy, 100 mg/10 ml), xylazine (Sigma Aldrich, Milan, Italy, 100 mg/10 ml) and acepromazine (prequillant, ATI Azienda Terapeutica Veterinaria Srl, 10 mg/10 ml) dissolved in saline solution. Animals were secured in a stereotaxic frame (David Kopf Instruments, Tujunga, CA, USA) and the peptide injection was accomplished in the lateral ventricle of mice at the following coordinates: AP=−0.2, ML=+1 e DV=2 relative to bregma, according to the atlas of Paxinos and Franklin [Paxinos G and Franklin K B J 2019]. The intracerebroventricular (icv) infusion was carried out by using a 25 μ1 Hamilton microsyringe connected to the infusion pump at constant flow rate of 2 μl/min for 1.30 min (volume injected 3 μl). The needle was left in place for other 3 min to avoid reflux. The control group (SHAM) received only vehicle, considering that, according to our previous observations [Morgese M G et al. 2017] the effects retrieved by the injection of reverse Aβ₄₂₋₁ were similar to the vehicle alone. At the time of dissection, the correct needle track was assessed. All in vivo and ex vivo experimental procedures were conducted in mice 7 days after surgery.

### βCP, FE and βBA Administration and Central Bioavailability Study

βCP, FE e βBA (Merck, Cayman Chemical Company) was dissolved in sunflower oil, this vehicle was chosen in order to maintain a better tasting and higher density of the final solution thus reducing reflux through the digestive tract. The dose of 5 mg/kg was chosen based on preliminary experiments indicating that this dose was the lowest showing antidepressant effects on intact mice (unpublished observations) and on other previously published data on animal models [Abdel-Tawab M et al. 2011].

Ten μl of the solution (or vehicle alone) were administered sublingually to mice. Brain βCP, FE e βBA were quantified in a separate subset of mice at 5, 15 and 30 min after their administration.

All behavioral experiments were conducted at 5 or 30 min after sublingual administrations of βBA or vehicle in mice treated with SHAM or AP. Based on the observed behavioral results, neurochemical and biochemical quantifications were performed 30 min after administration of βCP, FE, and βBA or vehicle in SHAM- or Aβ-treated mice.

### βCP, FE and βBA Quantification by GC-MS/IT

After administration, whole brains were put in 1 ml of a solution of chloroform/methanol (1:1 v/v), sonicated and then homogenates were centrifuged at 4° C. for 20 min (10,000 rpm). The pellets were then removed, and the remaining supernatants were dried on sodium sulphate anhydrous, filtered with 0.20 m PTFE syringe filters and used for chemical analysis. The quantification of βCP, FE and βBA was performed by using GC-MS/IT equipment composed of a gas chromatograph GC-7890B (Agilent Technologies, Santa Clara, CA, USA) coupled with an ion trap mass spectrometer IT-240 (Agilent Technologies). Mass data were acquired and worked out using the MS Workstation software version 8.0.1 (Agilent Technologies). All analyses were performed in triplicate.

### Behavioural Tests

Open Field Test. Mice were placed in an open field arena and allowed to explore for 30 min [Lama A et al. 2021]. After each trial, the arena floor was cleaned with 70% ethanol to avoid inter-assay bias. The movements of mice were video recorded and analyzed by ANY-maze tracking software (Ugo Basile-Varese, Gemonio, Italy). Locomotion was evaluated through total crossing measurements.

Splash test. The splash test was carried out as previously reported [Lama A et al. 2021]. A 10% sucrose solution was sprayed on the dorsal coat of the animal positioned alone in a Plexiglas cage (30×16×19 cm). The viscosity of the sucrose solution elicited robust self-grooming considered a self-care behavior. The test was videotaped and later an observer, blind to the trial, scored for latency to the first grooming event and duration of self-care behavior during the period of the test (5 min).

Tail Suspension Test. The test was performed according to Can A et al. 2012. Briefly, mice were left in the testing room 1 h prior to performing the test in order to allow for acclimation. Then animals were suspended by attaching their tails with adhesive tape (approximately 1 cm from the tip of the tail) to a suspension bar. The test was video recorded for 6 min, while immobility time was measured for the last 5 min. Immobility was considered when mice hung passively without moving.

### Post-Mortem Tissue Analysis

Anesthetized animals were sacrificed by cervical dislocation. Brains were immediately removed and kept on ice for dissection of PFC and HIPP, according to the atlas of Paxinos and Franklin. Tissues were frozen and stored at −80° C. until analyses were carried out. Samples (PFC and HIPP) were homogenized (1:10 p/v) at 4° C. using a PBS buffer containing a 1:100 protease and phosphatase inhibitor cocktail (HALT inhibitors, Thermo Fisher Scientific, Cleveland, OH, USA) for biochemical analyses or perchloric acid 0.1 M for neurochemical analyses. Homogenates were centrifuged at 13,000×g at 4° C. for 20 min, and supernatants were used for further determinations.

### Neurochemical Quantifications

5-HT, noradrenaline (NA), and KYN levels were measured in the PFC and HIPP of mice by HPLC coupled with an electrochemical detector (Ultimate ECD, Thermo Scientific Dionex, Milan, Italy) as already reported [Francavilla M et al. 2012, Morgese M G et al. 2016]. Separation was performed by a LC18 reverse phase column (Kinetex, 150 mm×3.0 mm, ODS 5 μm; Phenomenex, Castel Maggiore-Bologna, Italy). The detection was accomplished by a thin layer amperometric cell (Thermo Scientific Dionex, Milan, Italy) with a 5 mm diameter glassy carbon electrode at a working potential of 400 mV (5-HT and NA) or 0.750 mV (KYN) vs. Pd. The mobile phase consisted of an aqueous buffer containing 75 mM NaH₂PO₄, 1.7 mM octane sulfonic acid, 0.3 mM EDTA and acetonitrile 10%, buffered at pH 3.0. The flow rate was kept at 0.7 ml·min-1 by an isocratic pump (Shimadzu LC-10 AD, Kyoto, Giappone). Data acquisition and integration were performed by using Chromeleon software (version 6.80, Thermo Scientific Dionex, Milan, Italy) [Morgese M G et al. 2015, Morgese M G et al. 2016]. GLU concentrations were determined by HPLC coupled with fluorescence detection (emission length 460 nm; excitation length 340 nm), as previously published [Francavilla M et al. 2012]. Analyses were carried out using an LC18 reverse phase column (Kinetex, 150 mm×3.0 mm, ODS 5 m; Phenomenex, Castel Maggiore, Bologna, Italy) and detection was accomplished by pre-column derivatization with ophthalaldehyde/mercaptopropionic acid. The mobile phase consisted of a 50 mM sodium acetate buffer, at pH 6.95, with gradient methanol increasing linearly from 2 to 30% (v/v) over a 40 min run. The gradient flow rate was maintained by a pump (ASCO, Tokyo, Japan) at 0.5 ml/min. Results were analyzed by Borwin software (version 1.50; Jasco, Cremella, Italy) and the amino acid concentration was expressed as M. All data were normalized for total area weight and were expressed as concentration/mg of tissue.

### Western Blotting Quantification

The total amount of proteins was measured in homogenates by using Pierce BCA Assay (Thermo Fisher Scientific, Cleveland, OH, USA). Forty g of the total lysate protein were separated by SDS-PAGE precast gels (Bio-Rad Laboratories Inc., Segrate (MI), Italy), transferred onto nitrocellulose membranes (Bio-Rad Laboratories Inc, Segrate (MI), Italy) and then blocked for 1 h in blocking buffer (SigmaAldrich, Milan, Italy) [Schiavone S et al. 2017]. Rabbit poly-clonal antibody against GFAP (Dako Products, USA; 1:2,000), rabbit monoclonal antibody against CD11b (Abcam, Cambridge, MA, USA; ab133357, 1:1,000) and mouse monoclonal antibody against NF-kB μ65 (Santa Cruz Biotechnology, Dallas, Texas, USA; 1:2,000) were used to incubate the membranes overnight at 4° C. After HRP-conjugated specific antibody incubation, ECL reagent (Bio-Rad Laboratories Inc., Segrate (MI), Italy) was added to the immune complex and chemiluminescence was detected by ChemiDoc MP system (Bio-Rad Laboratories Inc., Segrate (MI), Italy). Optical densities of the bands were measured using Image J software (http://rsb.info.nih.gov/ij/ accessed on 15 Mar. 2021) and normalized against bands relative to -actin (1:5,000, Abcam, Cambridge, UK).

### Statistical Analyses

Data were expressed as mean±SEM. Experiments were analyzed using two-way (bioavailability data) or one-way analysis of variance (ANOVA) followed by Tukey's multiple comparisons test. AUCs data were analyzed by the unpaired Student's t-test. All analyses were performed by using GraphPad Prism 5 (GraphPad Software, San Diego, CA, USA). Differences among groups were considered significant at values of p<0.05.

### VII RESULTS

An iconographic summary of the results achieved with all four substances, alone, in pairs and all together, is shown, including the most representative graphs for each experimental group.

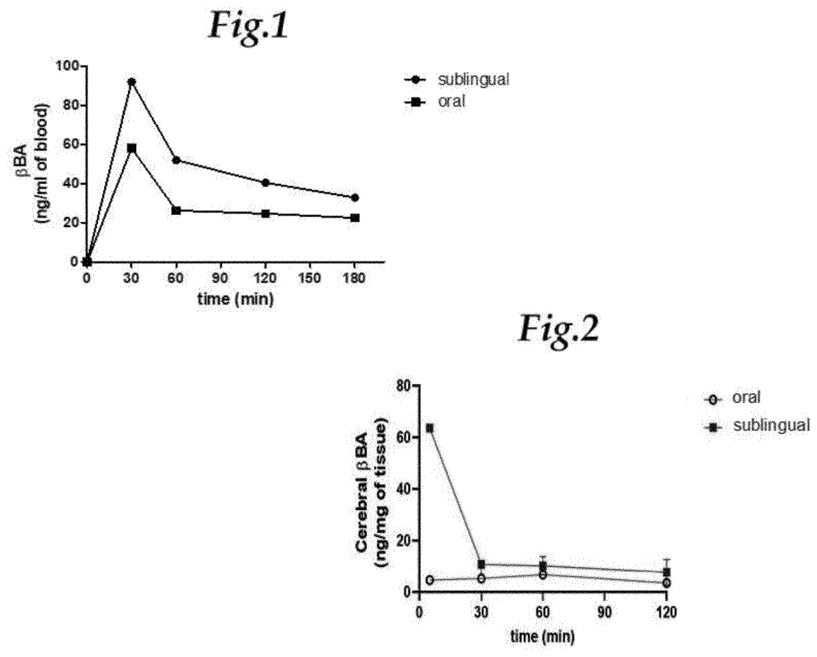

### 1. Administration of the Composition or Association of Compounds by Oral-Sublingual Vs Oral-Gastro-Intestinal Route (FIG. 1)

The administration by sublingual route, for the reasons already mentioned, allows a greater bioavailability especially of βBA, not water-soluble and with a higher molecular weight than the three terpenes of this compound.

By the sublingual route, in the present trial, pre-hematic loss of βBA was low, and sublingual βBA absorption was comparable to the enhancement obtained by hyperlipid meal via os [Sterk V et al. 2004]. In this case, sublingually, the blood concentration of βBA is reduced by 45% with respect to the administered dose, while via os the blood concentration is reduced by 70% with a sublingual gain of 46% (FIG. 1).

It is foreseeable that the association with the ω-3 DHA, fatty acid, contemplated in the composition or association object of the present invention, improves more the absorption of ß-boswellic acid.

FIG. 1. Blood Concentration of βBA Administered by Oral-Sublingual Route Vs. Oral-Gastro-Intestinal Route.
[symbol] 43 a, 58 kg BW
βBA administered both via os and sublingual: 10 mg=172.4 ng/g.
Maximum blood concentration via sublingual route: 95 ng/ml.
Maximum blood concentration via os: 52 ng/ml.
Δos/plasma: 1.82.
   Note
      1. The person tested with βBA after gastric ingestion (10 mg) did not experience any symptoms; sublingually, same measure, 10 minutes after she had a feeling of well-being at the occipito-parietal level, where she suffered from tension-type headache. The explanation is that βBA is an excellent vasodilator, probably through the release of NO nitric oxide [Wang M et al. 2015], synergistic with the other compounds of the composition or association object of the present invention.
      2. The experiment also gave indications regarding the dosage of ß-boswellic acid effective by the sublingual route, which is significantly lower than that administered via os according to literature data: twelve times lower than the dose given to healthy young volunteers by Sterk V et al. 2004, but 185 times lower than that used by Gerbeth K et al. 2011 in elderly patients. Note that with the present experiment, the Δos/plasma ratio of 1.82 was the same as that found by Sterk V et al. 2004 (Table 1).

### 2. Biotransformation of Composition or Association Via Sublingual Route to First Passage in the Brain (FIG. 2)

In addition to the dosage gain for the greater sublingual absorption, the composition or association at the first passage into the circulation, avoids the liver and reaches the brain (and other organs, such as the heart), where it can perform an effective vasodilating and metabolic action. In fact, a remarkable variety of enzymes are present both at the blood-blood-brain barrier level and in the brain tissue [Pavan B et al. 2008] where the composition or association can undergo intense biotransformation. This probably explains the reduction in blood levels, which we recorded in mice in the first minutes after the administration via sublingual of ß-boswellic acid (FIG. 2).

FIG. 2. Brain Tissue Concentration of βBA after Sublingual Administration Vs Via Os Administration. (Start Recording after 5 Min).

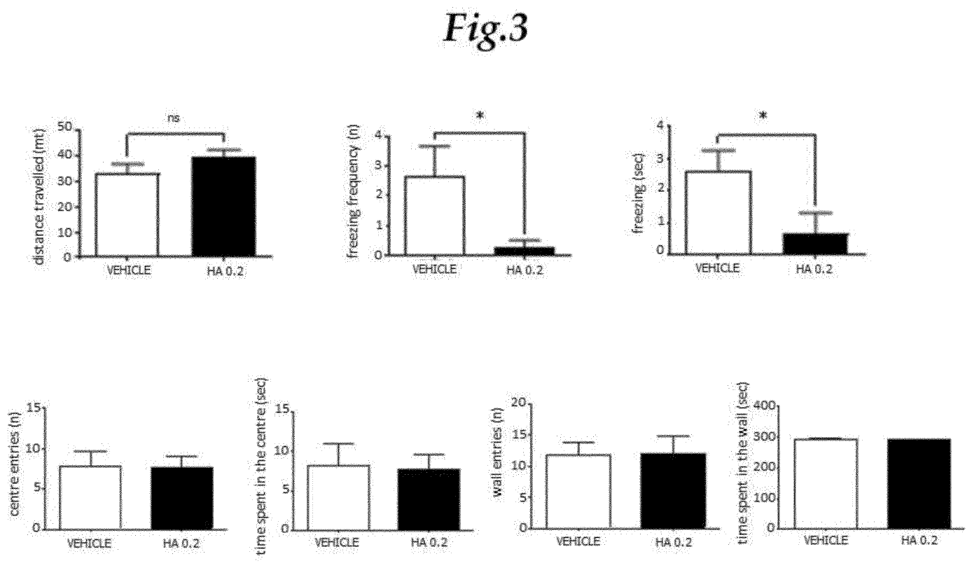

### 3. Individual Elements of the Composition or Association and Behavioral Tests on Intact Animals (FIG. 3)

The four components, HA₄, βCP, FE, βBA of the composition or association of compounds object of the invention have been both individually and in association shown to have an antidepressant and anxiolytic effect.

For example, we measured behavioral parameters in intact animals that received only hyaluronic acid. As can be seen from these graphs, HA₄ does not create deficits in the animal's locomotor activity, while it tends to have a clear anxiolytic and antidepressant effect (freezing data).

FIG. 3. Behavioral Testing with HA₄ in Intact Animals.

### FIG. 3. Anxiolytic and Antidepressant Action (Freezing) and Normal Motor Activity of Hyaluronic Acid Tetrasaccharide.

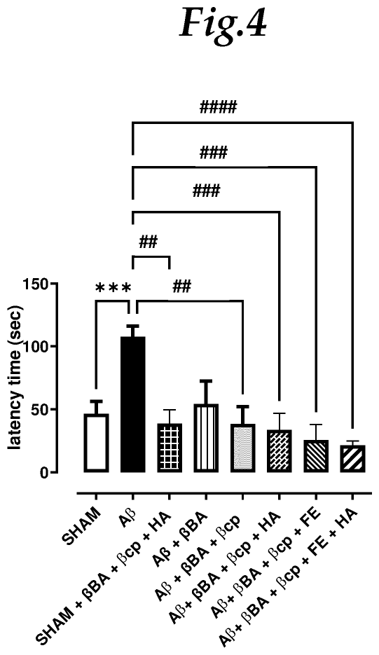

4. Elements of the Compound in Combination. Behavioral Tests in the Mouse Model Treated with Intracerebroventricular Aβ (icv). (FIG. 4)
FIG. 4. Behavioral Tests with HA₄+βCP+βBA and with HA₄+FE+CP+3BA in SHAM Mice and in Mice Treated with Aβ icv.
FIG. 4. Behavioral tests in SHAM mice and in mice treated with Aβ icv and after 7 days in a group with three substances (HA₄ 200 μg, βCP 50 μg, 3BA 100 μg) and in a second group with four substances of the composition (+FE 50 μg) sublingually. The antidepressant effect of the components of the composition, in combination, is very significant and synergistic both in intact animal (SHAM), and more so in mice after treatment with A3 icv.
The graph shows that the four components together maintain the antidepressant effect highlighted for 3CP and 3BA alone (data not shown), as well as the effect of combining the three components HA₄+βCP+βBA. Indeed, the addition of furaneoeudesma-1,3-diene has an even more significant synergistic antidepressant effect. The antidepressant effect is also evident against SHAM controls. Similarly, the reduced self-care time (data not shown) in the A3-only recipient group is also found to be back to the levels of the control mice after administration of the HA₄+3CP+3BA+FE combination. Legend: Splash test with latency time measurement (sec)

$$* p < 0.05$$

$$** p < 0 \text{.01}$$

$$\text{***} p < 0.001 ;$$

$$\text{***} SHAM vs A β$$

$$\text{***} SHAM + {HA}_{4} + β PC + β BA vs A β$$

$$\# p < 0.05$$

$$\#\# p < 0 \text{.01}$$

$$\#\# \# p < 0.001$$

$$\#\# \#\# p < 0.0001$$

$$\#\# \# A β + {HA}_{4} + β CP + β BA vs A β$$

$$\#\# \#\# \# A β + {HA}_{4} + β CP + FE + β BA vs A β$$

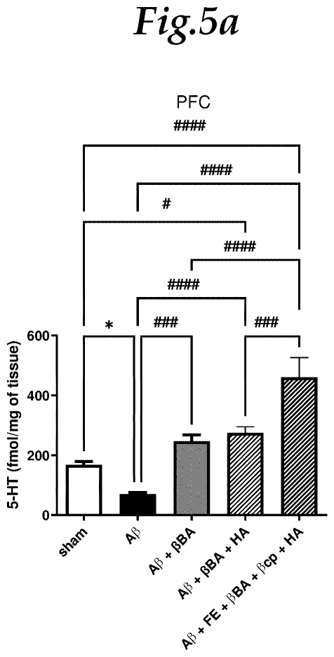

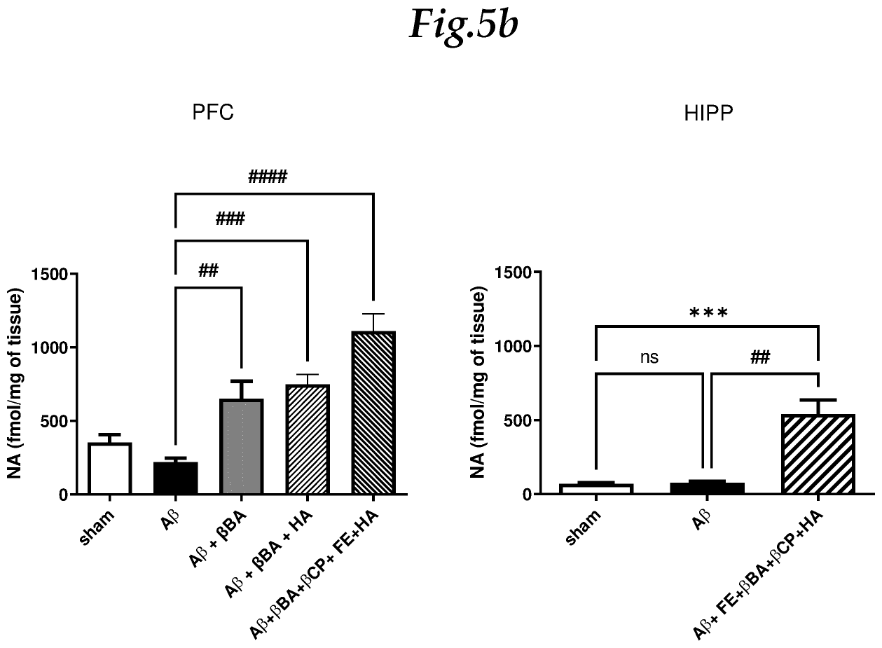

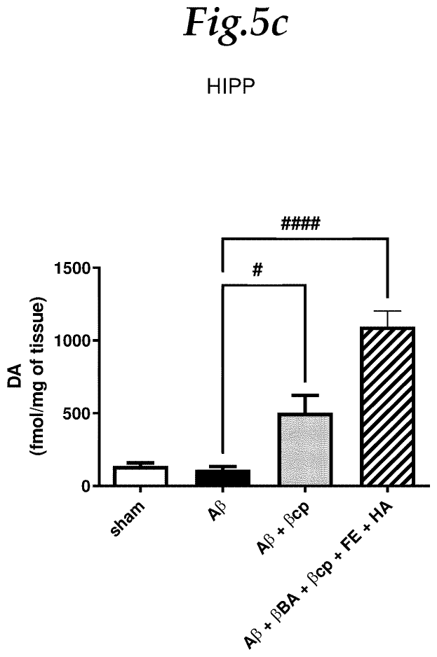

5. Elements of the Compound Alone and in Combination and Monoaminergic Neurotransmission. Anti-Anxiety and Antidepressant Action of the Compound. (FIG. 5a, 5b, 5c)

The significant increase in monoamines serotonin (5-HT), noradrenaline (NA), dopamine (DA) both in the prefrontal cortex (PFC) and in the hippocampus (HIPP) demonstrates the broad antidepressant and anti-anxiety activity of the composition or association of compounds of the present invention.

FIG. 5a. Serotonin (5-HT) Levels with βBA Alone, with βBA+HA₄ and with HA₄+CP+FE+.BA in Mice Treated with Aβ Icv.
FIG. 5a. Note the progressively greater serotonergic activity obtained by broadening the composition. Treatment with HA₄+CP+FE+βBA not only removes the reduction in 5-HT levels induced by A3 treatment, but also increases its levels compared with the control group

$$A β + β BA vs A β : p < 0.001$$

$$A β + β BA + {HA}_{4} vs A β : p < 0.001 but with less variability$$

$$A β + FE + β BA + β CP + {HA}_{4} vs A β : p < 0.0001 .$$

FIG. 5b. Norepinephrine (NA) levels with BA alone, with βBA+HA₄ and with HA₄+βCP+FE+βBA in mice treated with Aβ icv.
FIG. 5b. As can be seen from the two graphs, treatment with both βBA alone, both with βBA+HA₄, both with HA₄+3CP+FE+βBA removes the reduction of NA induced by Aβ treatment and increases its expression in both PFC and HIPP compared with the control group. Note the different expression of neurotransmitter in HIPP compared With PFC.

The associated action of serotonin and noradrenaline allows these active principles to reach the monoaminergic neurotransmitter systems in a greater number of brain areas [Stahl SM. Essential psychopharmacology. Cambridge Ed. 2021: 289-322]. A practical indication that the coexistence of a dual monoaminergic mechanism may lead to greater efficacy derives from the observation that venlafaxine, which is a serotonin-noradrenaline reuptake inhibitor, often appears to exhibit greater antidepressant efficacy with increasing dose, theoretically due to the gradual increasing inhibition of the noradrenaline transporter (ie, the so-called noradrenergic boost).

Since there is considerable overlap between the anxious and depressive symptoms, circuitry and neurotransmitters associated with anxiety disorders and those associated with major depressive disorder, it is not surprising that drugs developed as antidepressants have been shown to be effective in treating anxiety disorders. At present, the main treatments for anxiety disorders are always represented by drugs originally developed as antidepressants. Serotonin is a key neurotransmitter that interacts with the amygdala and with all elements of the cortico-striatum-thalamus-cortical circuit, such as the prefrontal cortex, the striatum and the thalamus, and is involved in the regulation of fear and excessive worry.

Antidepressants that can increase serotonin tone by blocking the serotonin transporter are also effective in reducing symptoms of anxiety and fear in all anxiety disorders, such as generalized anxiety disorder, panic disorder, social anxiety disorder (or social phobia) and post-traumatic stress disorder. These drugs are represented by the known selective serotonin reuptake inhibitors and serotonin-noradrenaline reuptake inhibitors [Stahl S M, ibidem].

FIG. 5c. Dopamine (DA) levels with βCP alone and with HA₄+βCP+FE+βBA in Aβ icv-treated mice.
FIG. 5c. Note the increase in dopaminergic activity in HIPP with β-caryophyllene significantly enhanced with HA₄+βCP+FE+βBA against the same SHAM controls.

Increased dopamine expression at the hippocampal level (not significant at the prefrontal level) may beneficially affect hedonic behavior, affective and cognitive symptoms as well as motor function (present experience on Parkinson's disease with boswellic acids).

Just with regard to DA levels in HIPP, the association of the 4 components significantly removes the reduction in DA levels. This effect deserves special attention taking into account that new “cognitive enhancers” (so-called cognitive enhancers) active on dopaminergic tone are under study precisely in AD.

The present preparation can precisely be called a “cognitive enhancer” and the invention of this is claimed.

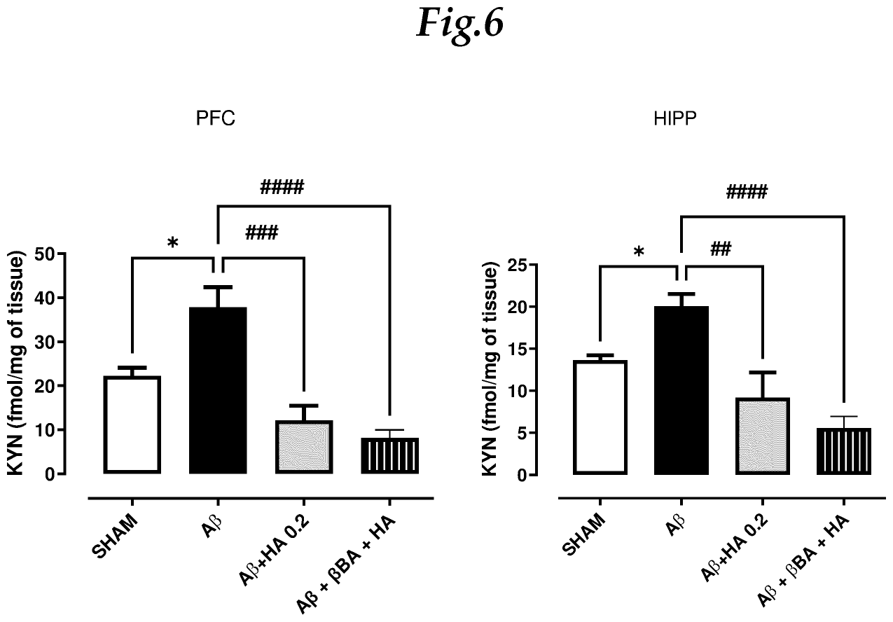

### 6. Compound Elements Alone or in Combination and Kynurenine (FIG. 6)

Kynurenine (KYN) is a molecule produced from tryptophan after enzymatic bioconversion by indolamine 2, 3-dioxygenase (IDO) enzymes. The metabolic shift from tryptophan metabolism to kynurenine and its derivatives, such as kynurenic or quinolinic acids, instead of 5-HT, has been proposed as another possible biological mechanism to explain a depressive state [Oxenkrug G 2013].

This research was done following our discovery that icv injection of Aβ was accompanied by an increase in kynurenine levels [Morgese M G et al. 2021], because a crucial crosstalk between the KYN pathway and glutamatergic function has been described [Schwarcz R 2016]. Therefore, we also investigated the role played by βCP, βBA, FE e HA₄ in regulating the interconnection between these biological substrates in the animal model treated with Aβ. The behavior of kynurenine in Aβ mice after treatment with HA₄ is shown as an example (FIG. 6). The reduction of kynurenine also occurred with the other elements of the compound and with the compound as a whole (graphs not shown because not significantly different).

FIG. 6. Kynurenine Levels with HA₄ Alone in Mice Treated with Aβ Icv.
FIG. 6. The two graphs show that following the insult with beta amyloid there is a significant increase in the levels of kynurenine in both areas considered, indicating the state of neuroinflammation. In mice treated with beta amyloid that received HA₄ at a dose of 0.2 mg/mouse there was a significant reduction in the levels of this molecule in the prefrontal cortex and in the hippocampus not only compared to the group that received beta amyloid but also towards the control group.

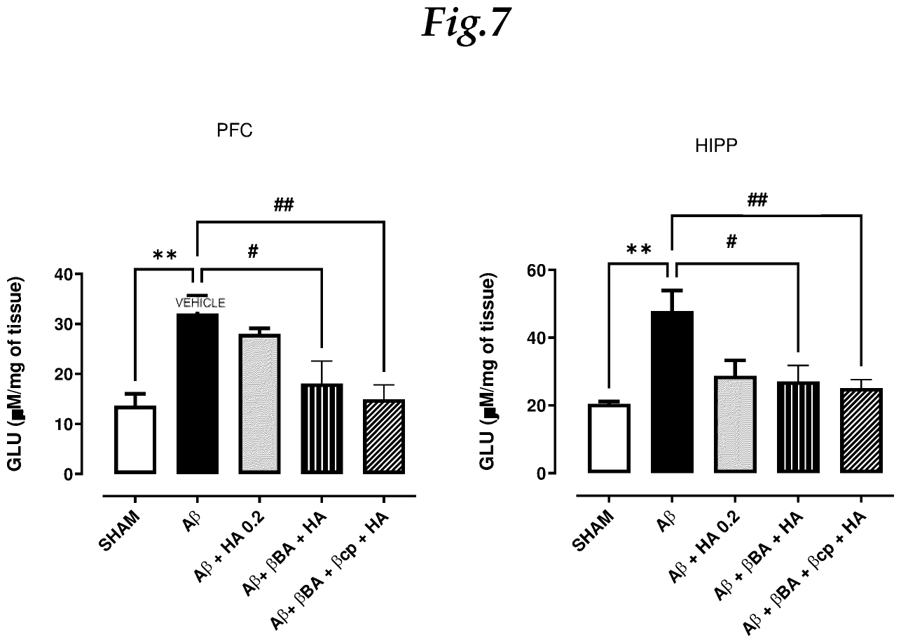

### 7. Composition or Association Concurs to Mitigate Excitotoxicity by Increasing Intracellular Ca²⁺ (FIG. 7)

In the present experience, following the insult with beta amyloid there is an increase in glutamate in both brain areas, more in HIPP, but without any significance of excitotoxicity. In fact, the compound in question, in particular its βBA component (not attached figure), reduces the mobilization of Ca²⁺ by increasing its intracellular concentration [Siemoneit U et al. 2017]; significantly reduces or restores the phlogosis indices represented in the other graphs (kynurenins, biomarkers of glial and astrocytic activation, nuclear factor NF-kB). Furthermore, the increase in glutamate induced by HA₄ especially in the hippocampus (one of the main stations of the glutamatergic circuits) seat of the memory system, probably has the significance of a long-term enhancement of learning, memory and neuroplasticity [Kandel E R et al. Principles of neural science. McGraw Hill; ed. VI, 2021:1339-69].

FIG. 7. Glutamate Levels with HA₄ Alone in Mice Treated with Aβ Icv.
FIG. 7. Brain tissue concentration of glutamate in the mouse model treated with Aβ icv and after 7 gg with HA₄. The increase in glutamate after insult with Aβ does not have significance of excitotoxicify but of contributing to the modulatory action of neuroinflammation by elevating intracellular Ca²⁺ and long-term potentiation of memory, learning, and neuroplasticity, considering that the hippocampus is the seat of the memory system.

### 8. Compound Elements Alone or in Combination and Biomarkers of Glial and Astrocytic Activation.

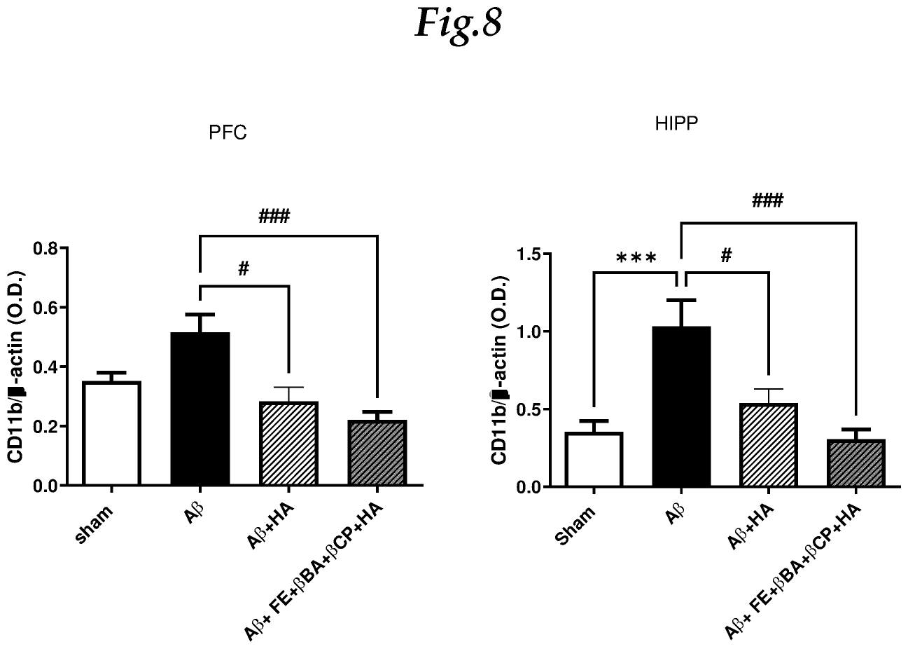

### I. CD11b (FIG. 8)

Quantifications of the CD11b protein levels present on the CD11b/CD18 receptor complex were performed that together they represent a microglial activation mechanism. In fact, CD11b is an integrin present on microglia, it represents the activation of microglia during neurodegenerative inflammation. As can be seen following the insult with beta amyloid, there is a significant increase in CD11b levels, indicating the state of microgliosis. In mice treated with amyloid beta that received HA₄ at a dose of 0.2 mg/mouse there was a significant reduction in levels of this protein in both the prefrontal cortex and in the hippocampus.

FIG. 8. CD11b Protein Levels with HA₄ in Mice Treated with Aβ Icv
FIG. 8. Brain tissue concentration of CD1 b protein levels in the mouse model treated with Aβ icv and after 7 days with HA₄ and With HA₄+FE+βCP+βBA. Significant reduction of CD11b in both PFC and HIPP.

### 9. Compound Elements Alone and in Association and Biomarkers of Glial and Astrocytic Activation.

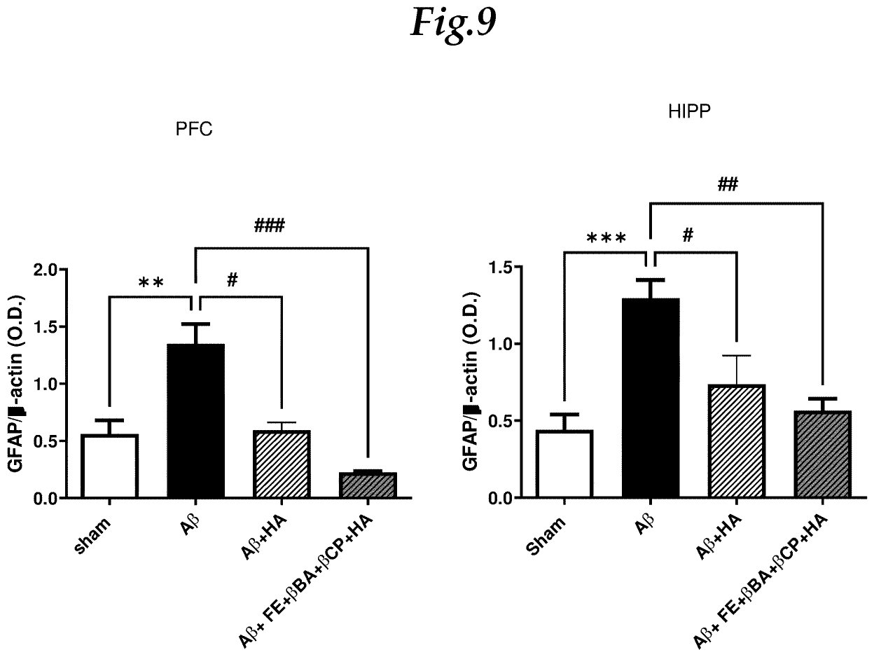

### II. GFAP (FIG. 9)

Quantifications of the protein levels of GFAP-acid fibrillar protein of the glia index of astrocytic activation were performed. As can be seen following the insult with beta amyloid, there is a significant increase in GFAP levels, indicating the state of astrogliosis. In mice treated with amyloid beta that received HA₄ at a dose of 0.2 mg/mouse there was a significant reduction in levels of this protein. This trend was maintained only partially in the hippocampus.

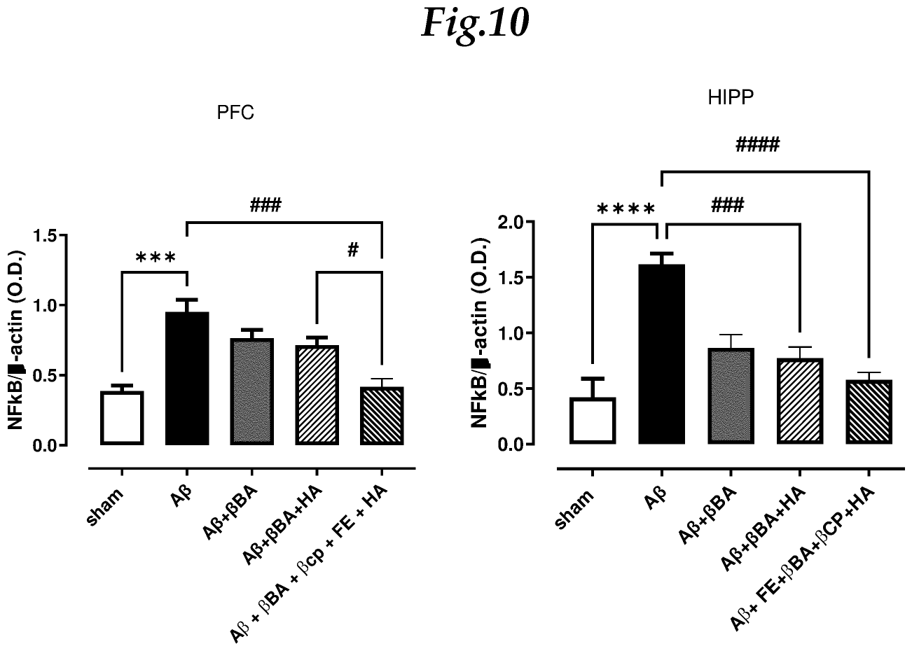

FIG. 9. Levels of GFAP Protein with HA₄ in Mice Treated with Aβ Icv.
FIG. 9. Brain tissue concentration of GFAP in the mouse model treated with A3 icv and after 7 days with HA₄ and with HA₄+FE+βCP+βBA. Administration of HA₄ and, with greater significance with HA₄+FE+βCP+βBA removes GFAP alterations in both PFC and HIPP, with less evidence in HIPP.
   10. Compound Elements Alone and in Combination and Western Blotting Analysis with Nuclear Factor NF-kB (FIG. 10)

NF-kB is a transcription factor that plays a primary role in the regulation of the immune response, in inflammation, in cell proliferation. It is also involved in the processes of synaptic plasticity and memory.

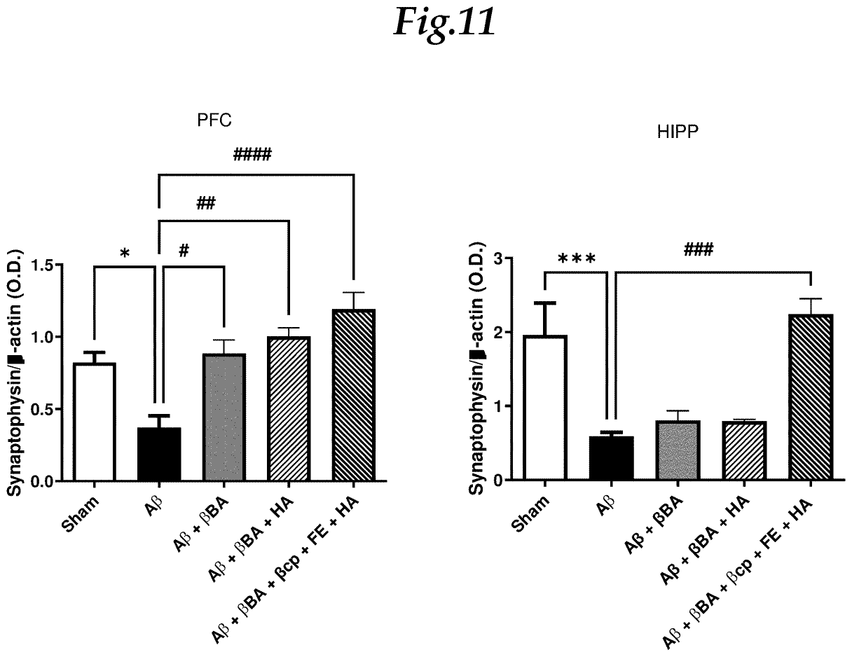

FIG. 10. Synergistic action of βBA, HA₄, βCP, FE on NF-kB.
FIG. 10. Tissue concentration in PFC and HIPP of NF-kB in three groups of mice treated with Aβ icv and after 7 days: with βBA; with βBA+HA₄; with βBA+HA₄+βCP+FE. There is a progressive removal of the pro-inflammatory effect induced by Aβ, which is more pronounced in HIPP.
   11. Compound Elements Alone and in Combination and Western Blotting Analysis with Synaptophysin (FIG. 11)

As mentioned above, this marker was chosen because synaptophysin is an integral membrane protein present at the level of the synaptic vesicle that has been studied regarding Aβ toxicity associated with NMDA glutamatergic receptor dysfunction.

### FIG. 11. Synergistic Action of βBA, HA₄, βCP, FE on Synaptophysin.

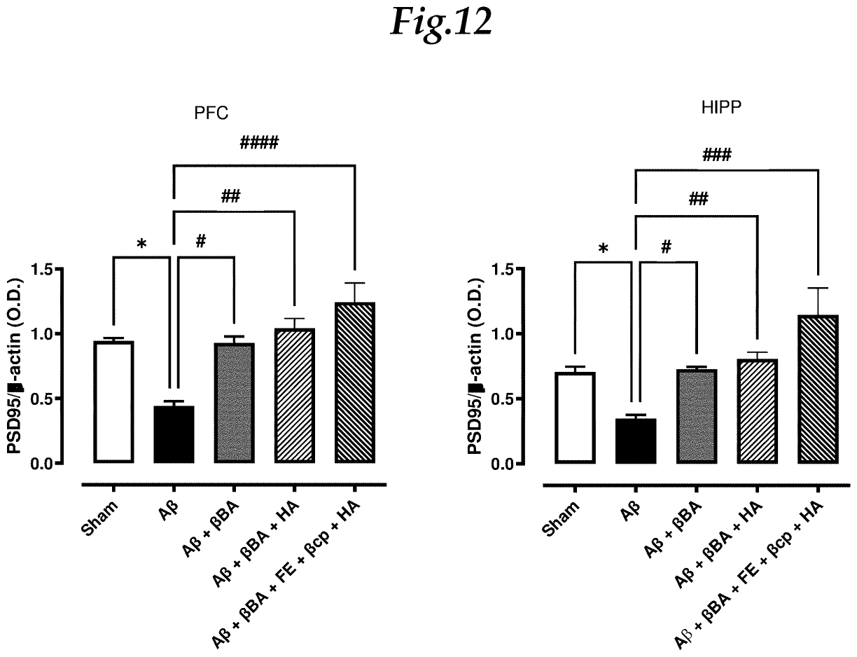

FIG. 11 Tissue concentration in PFC and HIPP of synaptophysin in three groups of mice treated with A3 icv and after 7 days: with βBA; with βBA+HA₄; with HA₄+FE+βCP+βBA. In all three groups, βBA, βBA+HA₄ and especially HA₄+FE+βCP+βBA remove Ap-induced alterations, most noticeably in PFC.
   12. Compound Elements Alone and in Combination and Western Blotting Analysis with PSD-95 (FIG. 12).

As already mentioned, this marker was chosen because PSD-95 is a postsynaptic protein that plays an important role in synapse maturation and synaptic plasticity and treatment with Aβ is known to lead to a reduction in the production of this protein.

FIG. 12. Synergistic action of βBA, HA₄, βCP, FE on PSD-95
FIG. 12. Tissue concentration in PFC and HIPP of PSD-95 in three groups of mice treated with A icv and after 7 days: with βBA, with βBA+HA₄ and with BA+HA₄+βCP+FE. The three compositions remove Aβ-induced alterations in a progressive manner, most pronounced with βBA+FE+βCP+HA₄ and most evident in PFC.

As shown in these graphs, treatment with both βBA, both βBA+HA₄, both HA₄+FE+βCP+βBA is progressively effective in modulating Aβ-induced synaptic dysfunction.

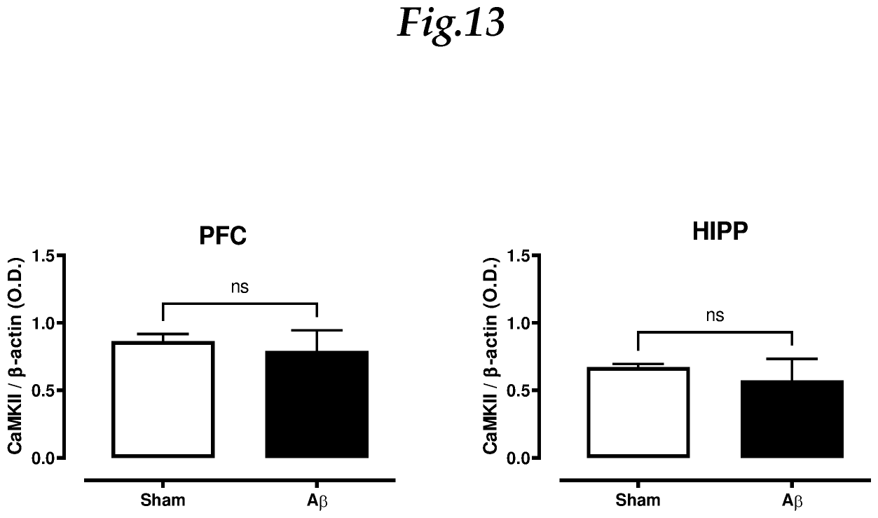

The effect is all the more significant when taking into account that it could be hindered by several mechanisms:
Anti-inflammatory action: proinflammatory states reduce both Synaptophysin and PSD-95 [Sheppard O et al. 2019].
Action on glutamate tone/astrocytic activation-deactivation.
Indirect anti-inflammatory action through reduction of glial activation.
   13. Western Blotting Analysis with CaMKII (FIG. 13)

CaMKII is a remarkably complex protein kinase known to play a key role in synaptic plasticity and memory formation. Furthermore, it has also been suggested that CaMKII is a tau kinase. CaMKII dysregulation may therefore be a modulator of toxicity in Alzheimer's disease.

The expression of Ca²⁺/calmodulin-dependent protein kinase II (CaMKII), a signal molecule fundamental for the trafficking and function of AMPA-type glutamate receptors, was quantified. This marker has been studied as previous studies indicated that the synaptic pool containing CaMKII was significantly reduced in cortical neurons of APP transgenic mice, mouse model of Alzheimer's, and the density of clusters containing CaMKII at the synaptic level was significantly reduced in mouse model which had received treatment with Aβ.

But for the CaMKII marker, treatment with Aβ in the present experimental conditions did not induce deficits for which we did not proceed with the evaluation of the effect of the elements of the composition (FIG. 13).

### FIG. 13. CaMKII Protein Levels in SHAM Controls vs Aβ Icv-Treated Mice.

FIG. 13. Treatment with A3 under the present experimental conditions did not induce CaMKII marker deficits, so we did not evaluate the composition.

### CONCLUSION

The summary judgment of the results obtained is that the compound object of this invention can represent a real step forward in the therapy of a broad spectrum of neurological and psychiatric disorders as specified in the claims.

1. A composition or association of compounds comprising:
a) the active principle docosahexaenoic acid DHA (C22:6 ω-3 C₂₂H₃₂O₂ MW 328.488) mixed with all or in part, with:
b) hyaluronic acid HA₄ tetrasaccharide (C₂₈H₄₄N₂O₂₃ MW 776) in nanoparticles
c) β-caryophyllene βCP (C₁₅H₂₄ MW 204.35),
d) furanoeudesma-1,3-diene FE (C₁₅H₁₈O MW 214.30),
e) β-boswellic acid βBA (C₃₀H₄₈O₃ MW 456.7).

2. The composition or association of compounds according to claim 1, comprising three compounds of HA₄ nanoparticles aggregated with βCP, FE, OBA with an electrostatic field system mixed with ω-3 DHA.

3. The composition or association of compounds according to claim 1, comprising nanoparticles of HA₄ aggregated with sesquiterpene βCP, mixed with ω-3 DHA.

4. The composition or association of compounds according to claim 1, of HA₄ nanoparticles aggregated with sesquiterpene FE, mixed with ω-3 DHA.

5. The composition or association of compounds according to claim 1, of HA₄ nanoparticles aggregated with pentacyclic triterpene βBA, mixed with ω-3 DHA.

6. The association according to claim 1, of two compounds of HA₄ nanoparticles aggregated with the two sesquiterpenes βCP and FE, mixed with ω-3 DHA.

7. The association according to claim 1, of two compounds of HA₄ nanoparticles aggregated with sesquiterpene βCP and triterpene βBA, mixed with ω-3 DHA.

8. The association according to claim 1, of two compounds of HA₄ nanoparticles aggregated with sesquiterpene FE and triterpene βBA, mixed with ω-3 DHA.

9. The composition or association of compounds according to claim 1, characterized by providing that in the product the final concentration of HA₄ in the solution is 40-80 mg/ml; that of βBA terpene is 20-40 mg/ml; those of FE and βCP are 10-20 mg/ml.

10. The composition or association of compounds according to claim 1, for use in treating morbid conditions such as, for example, dementia, demyelinating and movement disorders, headaches wherein the composition or association is administered in adult humans at a therapeutic dose of hyaluronic acid HA₄ tetrasaccharide which is in the range of 40-80 mg/day and the range may vary from 0.40 to 1.20 mg/kg body weight.

11. The composition or association of compounds according to claim 1, for use in treating morbid conditions such as, for example, dementia, demyelinating and movement disorders, headaches wherein the composition or association is administered in adult humans at a therapeutic dose of the terpene β-boswellic acid βBA which is around 20-40 mg/day and the range may vary from 0.20 to 0.60 mg/kg body weight.

12. The composition or association of compounds according to claim 1, for use in treating morbid conditions such as, for example, dementia, demyelinating and movement disorders, headaches wherein the composition or association is administered in adult humans at a therapeutic dose of the terpene furanoeudesma-1,3-diene which is around 10-20 mg/day and the range may vary from 0.10 to 0.30 mg/kg body weight.

13. The composition or association of compounds according to claim 1, for use in treating morbid conditions such as, for example, dementia, demyelinating and movement disorders, headaches wherein the composition or association is administered in adult humans at a therapeutic dose of the terpene (3-caryophyllene βCP which is around 10-20 mg/day and the range may vary from 0.10 to 0.30 mg/kg body weight.

14. The composition or association of compounds according to claim 1, for use in treating morbid conditions such as, for example, dementia, demyelinating and movement disorders, headaches wherein the composition or association is administered in adult humans at a therapeutic dose of the docosahexaenoic acid DHA of at least 400 mg/day and the range may vary from 5 to 12 mg/kg body weight.

15. The composition or association of compounds according to claim 1, obtained by the combined method of rotating magnetic field associated with another well-known method of electrostatic field system with the achievement of a higher density of HA₄ nanoparticles aggregated with βBA, FE, βCP in electrostatically stable aqueous solution at room temperature.

16. A method for administering the composition or association of compounds according to claim 1 sublingually aimed at obtaining a nutraceutical product by any means (e.g. drops, sugared almonds, nanoconstructs) according to the pathophysiology of the disease to be treated.

17. A method for administering the composition or association of compounds according to claim 1 via os aimed at obtaining a nutraceutical product by any means (e.g. tablets, suspensions, sachets, nanoconstructs) for gastro-intestinal diseases.

18. A method for administering the composition or association of compounds according to claim 1 via os aimed at obtaining a nutraceutical product by any means (e.g. tablets, suspensions, sachets, nanoconstructs) for systemic diseases.

19. A method for administering the composition or association of compounds according to claim 1 nasally by any means (e.g. aerosol, spray, drops, nanoconstructs) according to the pathophysiology of the disease to be treated.

20. A method for administering the composition or association of compounds according to claim 1 by pulmonary route by any means (e.g. aerosol, spray, endotracheal instillations, nanoconstructs) for respiratory and systemic diseases.

21. A method for administering the composition or association of compounds according to claim 1 parenterally (intravenously, intramuscularly, subcutaneously) or endoviscerally (e.g. endovesical, endorectal) or endo-articularly, by any means (e.g. nanoconstructs, solutions, suppositories).

22. A method for administering the composition or association of compounds according to claim 1 by cutaneous, mucosal, intradermal route with any means realized for curative or aesthetic purposes (e.g. nanoconstructs, hydrogels, ointment, cream, patches, infiltration).

23. The composition or association of compounds according to claim 1, for use in prevention, in treatment, as a cognitive enhancer, or as an adjuvant, or as a supplement in disorders and diseases of the Nervous System, according to the pathophysiology of the disease to be treated:
Degenerative diseases: progressive dementias (e.g. Alzheimer's disease, Pick's disease), Down syndrome.
Alterations in posture and movement (e.g. Parkinson's disease, Huntington's chorea).
Progressive spino-cerebellar, cortico-cerebellar ataxias, involving the brain stem and other neurological disorders.
Motor disorders with amyotrophy (e.g. amyotrophic lateral sclerosis).
Spastic paraplegias without amyotrophy (e.g. primary lateral sclerosis).
Sensory and sensory-motor disorders (e.g. Dejerine-Sottas polyneuropathy).
Neurological disorders associated with alcohol.
Progressive blindness (eg, retinitis pigmentosa), progressive ophthalmoplegia (e.g., Kearns-Sayre syndrome).
Genetic hypoacusis, sensorineural (sensorineural deafness) and central (central deafness).
Hyposmia, anosmia, dysosmia, parosmia, olfactory agnosia.

24. The composition or association of compounds according to claim 1, for use in prevention, in treatment or as an adjuvant or as a supplement in disorders and diseases of the Nervous System, according to the pathophysiology of the disease to be treated
Demyelinating diseases, e.g. multiple sclerosis, optic neuromyelitis, disseminated post-infectious and post-vaccine encephalomyelitis, hereditary and demyelinating cerebellar ataxias
Diseases of the spinal cord: e.g. infectious and non-infectious inflammatory diseases, vascular diseases, subacute or chronic spinal paraparesis syndrome with or without ataxia.
Diseases of the peripheral nerves: polyneuropathy, radiculopathy, neuronopathy, plexopathy; hereditary peripheral neuropathies; Dercum's disease; tremors, myoclonus, spasms and tics.
Restless Legs Syndrome; flaccid newborn syndrome.
Diseases of the cranial nerves (e.g. neuralgia, trigeminal herpes zoster, facial paralysis, multiple paralysis).
Multiple sclerosis optic neuropathy, radiation, hereditary and developmental anomalies; post-infectious and viral neuroretinitis; papillary edema.

25. The composition or association of compounds according to claim 1, for use in prevention, in treatment or as an adjuvant or as a supplement in disorders and diseases of the Nervous System, according to the pathophysiology of the disease to be treated:
Headaches in many types: migraine without aura, migraine with aura, cluster headache, tension headache, temporal arteritis and their variants.
Vascular, viral, toxic and anxious vertiginous syndromes, vestibulopathies, balance disorders.
Epilepsies in multiple clinical characteristics and variants: somatic motor, somato-sensitive, auras, hallucinations, illusions, absences, bilateral epileptic myoclonus, Lennox-Gastaut syndrome.
Post-hypoxic neurological syndromes.
Prevention of cerebral ischemic attacks and the cardiovascular system.
Acquired metabolic diseases: e.g. diabetic or renal acidosis, hepato-cerebral degeneration, steroid encephalopathy.

26. The composition or association of compounds according to claim 1, for use in prevention, in treatment or as an adjuvant or as a supplement in disorders and diseases of the Nervous System, according to the pathophysiology of the disease to be treated, in all their clinical expressions:
Mood and emotional disorders: chronic fatigue, asthenia, anxiety, panic attacks, hyperventilation syndrome, depression.
Disorders involving limbic structures: perceptual and cognitive alterations;
emotional lability; pathological laughter and crying; anger and aggression reactions; fear; apathy and placidity; frontal and thalamic syndromes; euphoria.
Psychic eating disorders: anorexia, bulimia, obesity.
Speech and language disorders in the various syndromic forms.
Severe mental retardation in its various forms (dysmorphic defects, learning and developmental disorders such as intellectual disability, dyslexia, Rett syndrome;
mental disorders in children and adolescents)
Psychiatric disorders: phobic neurosis, obsessive-compulsive neurosis, hysteria in various manifestations, hypochondria.
Personality disorders (e.g. paranoid, cyclothymic, schizoid, antisocial or sociopathy).
Autism (childhood autism, Kanner-Asperger syndrome, autistic range disorders).
Schizophrenia, paranoid states, puerperal psychosis.
Reactive depression, endogenous depression, manic-depressive psychosis, unipolar depression, bipolar depression, suicide risk.

27. The composition or association of compounds according to claim 1, for use in prevention, in treatment or as an adjuvant or as a supplement in acute and chronic infectious diseases with neurological involvement, in particular from:
Neurotropic viruses: cytomegalovirus encephalitis, chicken pox, herpes zoster and simplex viruses; myelitis, polyradiculopathies.
HIV: acute, subacute and chronic encephalitis; HIV-dementia complex, polyradiculopathies.
SARS-COV-2: hyposmia and anosmia; Alzheimer-Parkinson-multiple sclerosis; COVID 19-dementia complex, including central and peripheral neurological complications and long-term sequelae.
Prions: subacute spongiform encephalitis in its forms of Creutzfeld-Jacob disease, Gerstmann-Straussler-Scheinker syndrome, fatal familial insomnia.

28. The composition or association of compounds according to claim 1, for use in prevention, in treatment or as an adjuvant or as a supplement in psychic disorders of female sexuality (sexual desire/interest disorders, sexual arousal disorders, orgasm disorders, vaginismus, dyspareunia, postpartum depression) and of male sexuality (reduced libido, ejaculation precox, erectile disorder, impotence) and in infertility due to hypospermia and deficiency of spermatic and ciliary tubal motility.

29. The composition or association of compounds according to claim 1 for use:
As an adjuvant or as a supplement in immune and stem cell therapy of central and peripheral nervous system diseases.
In prevention, in treatment or as an adjuvant or as a supplement in other diseases with involvement in the Central Nervous System of hyaluronic acid, the CB₂ endocannabinoid system and the μ-receptors of the opioid system.
